(19) United States
(12) Patent Application Publication
(10) Pub. No.: US 2024/0287553 A1
(43) Pub. Date: Aug. 29, 2024

(54) Methods of Production of Double Unsaturated Pheromones Using Metabolic Pathways

(71) Applicants: ISCA Technologies, Inc., Riverside, CA (US); SEMIOPLANT AB, Lund (SE)

(72) Inventors: Jan Christer Löfstedt, Lund (SE); Per Fredrik Hofvander, Bjärred (SE); Binu Antony, Riyadh (SA); Honglei Wang, Lund (SE); Baojian Ding, Lund (SE); Agenor Mafra-Neto, Riverside, CA (US)

(21) Appl. No.: 18/492,600

(22) Filed: Oct. 23, 2023

Related U.S. Application Data
(63) Continuation of application No. 18/055,229, filed on Nov. 14, 2022, now abandoned.
(60) Provisional application No. 63/305,104, filed on Jan. 31, 2022.

(51) Int. Cl. C12P 7/62; C12N 9/02; C12N 9/10; C12N 15/81

(52) U.S. Cl. CPC C12P 7/62; C12N 9/0008; C12N 9/0071; C12N 9/1029; C12N 15/81

(57) ABSTRACT

ZETA can be produced by genetically modifying a genome to incorporate a gene encoding an E12 fatty acyl desaturase and then providing a fatty acid. The gene encoding an E12 fatty acyl desaturase can be incorporated into a yeast or plant genome.

### CROSS-REFERENCE TO RELATED APPLICATIONS

This application claims the benefit of U.S. patent application Ser. No. 18/055,229, filed on Nov. 14, 2022, which also claims the benefit of U.S. Provisional Application No. 63/305,104, filed on Jan. 31, 2022, and U.S. Provisional Application No. 63/263,996, filed on Nov. 12, 2021, the teachings of which are both expressly incorporated by reference.

### STATEMENT RE: FEDERALLY SPONSORED RESEARCH/DEVELOPMENT

Not Applicable

### REFERENCE TO AN ELECTRONIC SEQUENCE LISTING

The contents of the electronic sequence listing (ISCA_042 US.xml; Size: 19,666 bytes; and Date of Creation: Nov. 14, 2022) is herein incorporated by reference in its entirety.

### BACKGROUND

The present disclosure relates generally to methods of producing pheromones using metabolic pathways, and more particularly to methods of creating (Z,E)-9,12-tetradecadienyl acetate, also known as Z9,E12-14:OAc or “ZETA”.

In general, ZETA is a major sex pheromone component for many stored-product moth species, as well as a sex pheromone component of global agricultural pests, such as beet armyworm (Spodoptera exigua), Egyptian cotton leafworm (Spodoptera littoralis), and tobacco cutworm (Spodoptera litura). ZETA is used worldwide for mating disruption, detection, monitoring, and mass trapping in raw and processed food storage facilities. The present disclosure recites a novel way of producing ZETA pheromone and its precursor biologically, using biobricks from various sources to successfully assemble the biosynthetic pathway in the engineered yeast Saccharomyces cerevisiae and a plant Nicotiana benthamiana, demonstrating the feasibility of producing ZETA by metabolic engineering.

Worldwide, cosmopolitan stored product insect pests inflict uncountable damage and economic losses on stored products such as maize, wheat, and other grains, dates, chocolates, dried cocoa beans, dried fruits, beans, nuts, tobacco, coconut, bananas, and groundnuts in stores. Among these pests, moths belonging to the family Pyralidae (lepidoptera) contaminate and destroy almost all aforesaid stored food in factories and warehouses. Noticeably, the Indian meal moth, Plodia interpunctella (Hübner), Mediterranean flour moth (Ephestia kuehniella Zeller), and the almond moth (tropical warehouse moth) Ephestia (Cadra)cautella (Walker) are highly destructive and cause a substantial increase in economic costs for pest control, quality loss, and dissatisfaction of consumers. Larvae of all species possess glands that secrete silk with which they interlink food products as they move. A considerable amount of damage results from webbing in the grain and on the surface of bags forming large lumps; therefore, food is no longer fit for consumption once infested. In the past, the control of these pests depended entirely on pesticides and fumigants, mainly using two universally available fumigants, viz., methyl bromide and phosphine (PH3). However, there are increasingly strict regulations on using such chemicals; methyl bromide faced a worldwide phase-out by 2015 under the terms of the Montreal Protocol.

The use of insecticides to control insect populations is increasingly difficult due to governmental regulations, off-target impact on beneficial species, harmful human health and environment, and evolution of insecticide resistance. For example, insecticide residue in the fresh and storage date fruit has been a significant concern in the dates industry in Middle Eastern countries in recent years, as importing countries have imposed stringent regulations on permissible pesticide residues. As an alternative to conventional pesticides, Integrated Pest Management (IPM), including mating disruption (MD) and mass trapping with sex pheromones to prevent insect reproduction, is considered one of the most promising and scalable solutions. The major sex pheromone component for the abovementioned pyralid moths is (Z,E)-9,12-tetradecadienyl acetate (ZETA, Z9,E12-14:OAc). This pheromone is currently commercially available and used worldwide. Besides, there is a high use of ZETA pheromone for MD of other key agricultural pests globally, such as bect armyworm (Spodoptera exigua Hübner), Egyptian cotton leafworm (Spodoptera littoralis Boisduval), and tobacco cutworm (Spodoptera litura Fabricius). Like many commercially available pheromones, large amounts of ZETA are currently produced by chemical synthesis, which requires expensive and often hazardous specialty chemicals as starting materials and may result in toxic waste as byproducts. Hence, pest management in warehouse and storage facilities using ZETA pheromone is costly and has millions of dollars spent annually. To account for the increased demand for the ZETA pheromone worldwide, a cost-effective and safe alternative method to synthetic chemical production is needed.

Moth sex pheromones, in general, are fatty acid derivatives produced de novo in the pheromone gland with 0-4 double bonds in the acyl chain and an oxygenated functional group such as aldehyde, alcohol, and acetate ester. Sex pheromones or sex attractants have been identified for thousands of moth species (PheroBase.com). Significant advances have been made during the last 20 years to understand the molecular basis of moth pheromone biosynthesis. The biological production of several monounsaturated moth sex pheromone components has been achieved in both yeast and plant recently. High titre of the cotton bollworm and the fall armyworm pheromone precursors in the form of corresponding pheromone alcohols were achieved through fermentation of an oleaginous yeast Yarrowia lipolytica upon systematic metabolic engineering. A similar approach was used to make the corresponding alcohols of the pheromone of European corn borer in Y. lipolytica.

In the present disclosure, we demonstrate the feasibility of producing the ZETA pheromone component in yeast Saccharomyces cerevisiae by the concerted expression of a suite of biosynthetic enzymes. First, we functionally characterized a unique 412 desaturase essential for the biosynthesis of the ZETA pheromone. We then used biobricks from various sources to successfully assemble the biosynthetic pathway in the yeast, demonstrating the feasibility of producing ZETA by metabolic engineering. Furthermore, we tested the biological activity of the yeast-produced ZETA by gas chromatography coupled to an electroantennographic detector (GC-EAD), using antennae of male S. exigua and P. interpunctella, of which species the females produce ZETA as their major sex pheromone component. We have shown for the first time that a doubly unsaturated acetate sex pheromone can be produced in yeast and pave the way for green production of an extended portfolio of moth sex pheromones for integrated pest management.

### BRIEF SUMMARY

In the present disclosure, the pheromone gland (PG) transcriptome data of the almond moth, Ephestia (Cadra)cautella (Walker) was mined to trace a novel E12 fatty acyl desaturase and expressed candidates heterologously in yeast and Sf9 systems. Further, it was demonstrated that a tailor-made ZETA pheromone bioproduction in yeast and plants through metabolic engineering using this E12 desaturase, in combination with three genes from various sources coding for a Z9 desaturase, a fatty acyl reductase, and an acetyltransferase, respectively, is possible. Electrophysiological assays (gas chromatography coupled to an electroantennographic detector, GC-EAD) proved that the transgenic yeast-produced ZETA pheromone component elicits distinct antennal responses. As such, the reconstructed biosynthetic pathway in yeast and plants shown below efficiently produces ZETA pheromone, leaves an undetectable level of biosynthetic intermediates, and paves the way for the economically competitive high-demand ZETA pheromone's bioproduction technology for high-value storage pest control.

In particular, a novel 412 desaturase, Ecau_D4_ASVQ, with unique biological activity was isolated and functionally analyzed. This desaturase is useful for the heterologous production of the final ZETA, Z9,E12-14:OAc pheromone in Saccharomyces cerevisiae. The Saccharomyces cerevisiae olel/elol disruption strain is useful for the production of ZETA, Z9, E12-14:OAc, when fatty acid precursors are fed to the yeast. For production of ZETA, Z9, E12-14:OAc from simple substrates the Ecau_D4_ASVQ expression in yeast is complemented by the expression of Dmel_D9, Sexipg-FARII and ATF1. For production of Z9E12-14:acid in vegetative plant tissue, integrated in the plant genome is one or several copies of a 14-16 carbon fatty acid specific thioesterase gene, one or several copies of a Dmel_D9 desaturase gene and one or several copies of a Ecau_D4_ASVQ desaturase gene all under the control of promoter elements for vegetative tissue expression. For production of Z9E12-14:acid as a component of seed oil in an oilseed plant, integrated in the plant genome is one or several copies of a 14-16 carbon fatty acid specific thioesterase gene, one or several copies of a Dmel_D9 desaturase gene and one or several copies of a Ecau_D4_ASVQ desaturase gene all under the control of promoter elements for seed expression. Moreover, the Z9E12-14C can be incorporated to wax esters by introducing one or several copies of the SexipgFARII gene and one or several copies a wax synthase gene selected from MhyWS or SauWS. For production of ZETA, Z9,E12-14:OAc in vegetative plant tissue integrated in the plant genome is one or several copies of the Sexi_pgFARII and one or several copies of the ATF1 gene can be combined in addition to one or several copies of a 14-16 carbon fatty acid specific thioesterase gene and one or several copies of desaturase Dmel_D9 and Ecau_D4_ASVQ genes, all under the control of promoter elements for vegetative tissue expression.

In accordance with one embodiment of the present disclosure, there is contemplated a method of producing (Z,E)-9,12-tetradecadienyl acetate. The method includes genetically modifying a genome of a species to incorporate into the genome a gene encoding an E12 fatty acyl desaturase and providing a fatty acid to the species. The fatty acid may be a fatty acid methyl ester. The fatty acid methyl ester may be myristic acid methyl ester. The species may be Saccharomyces cerevisiae.

The gene encoding an E12 fatty acyl desaturase may be the gene of SEQ ID NO: 2. The genome may further incorporate a gene encoding a Z9 desaturase, a gene encoding a fatty acyl reductase, and a gene encoding an acetyltransferase. The gene encoding a Z9 desaturase may be Dmel_D9 from Drosophila melanogaster, the gene encoding a fatty acyl reductase may be SexipgFARII from Spodoptera exigua, and the gene encoding an acetyltransferase may be ATF1 from Saccharomyces cerevisiae.

Another embodiment of the present disclosure contemplates a genetically modified yeast having incorporated into the genome a gene encoding an E12 fatty acyl desaturase, wherein the yeast produces a pheromone. The gene encoding an E12 fatty acyl desaturase may be the gene of SEQ ID NO: 2. The E12 fatty acyl desaturase may be the peptide of SEQ ID NO: 8. The genome may further incorporate a gene encoding a Z9 desaturase, a gene encoding a fatty acyl reductase, and a gene encoding an acetyltransferase. The gene encoding a Z9 desaturase may be Dmel_D9 from Drosophila melanogaster, the gene encoding a fatty acyl reductase may be SexipgFARII from Spodoptera exigua, and the gene encoding an acetyltransferase may be ATF1 from Saccharomyces cerevisiae. The yeast may be Saccharomyces cerevisiae, and the pheromone may be (Z,E)-9,12-tetradecadienyl acetate.

Yet another embodiment of the present disclosure contemplates a genetically modified plant having incorporated into the genome a gene encoding an E12 fatty acyl desaturase, wherein the plant produces a pheromone. The gene encoding an E12 fatty acyl desaturase may be the gene of SEQ ID NO: 2. The E12 fatty acyl desaturase may be the peptide of SEQ ID NO: 8. The genome may further incorporate a gene encoding a Z9 desaturase, a gene encoding a fatty acyl reductase, a gene encoding an acetyltransferase, and a gene encoding a 14-16 carbon fatty acid specific thioesterase. The gene encoding a Z9 desaturase may be Dmel_D9 from Drosophila melanogaster, the gene encoding a fatty acyl reductase may be SexipgFARII from Spodoptera exigua, and the gene encoding an acetyltransferase may be ATF1 from Saccharomyces cerevisiae. The plant may be Nicotiana benthamiana, and the pheromone may be (Z,E)-9,12-tetradecadienyl acetate.

### BRIEF DESCRIPTION OF THE DRAWINGS

These and other features and advantages of the various embodiments disclosed herein will be better understood with respect to the following description and drawings, in which like numbers refer to like parts throughout, and in which:

FIG. 1 shows the expression levels of desaturases in the pheromone gland of E. cautella. The mean relative fold-change expression of different desaturase genes was measured from raw cycle threshold (Ct) values relative to the expression of the endogenous control genes, viz., actin and elongation factor, as recited in Table 1.

FIG. 2 shows the phylogeny of desaturase genes. The maximum likelihood tree was constructed using amino acid sequences of 122 fatty acyl desaturase genes of 30 insect species. The E. cautella desaturases are indicated by a solid dot. Desaturase groups with similar activities are indicated in parenthesis, and bootstrap values are shown at the node of each branch. The arrows indicate the A11 desaturases involved in E. cautella pheromone biosynthesis.

FIG. 3 shows the GC-MS analysis of FAMEs from yeast expressing Ephestia cautella desaturase supplemented with 12:Me* and 14:Me*. FAMEs of (a) control yeast transformed by empty vector; (b) Ecau_Des2_RPVE; (c) yeast expressing Ecau_Des6_NPVE; (d) Ecau_Des9_KPSE; (c) Ecau_Des4_ASVQ; (f) Ecau_Des11_VPVQ and (g) Ecau_Des14_LPVQ. DMDS derivatives of FAME from yeast expressing Ecau_D11_VPVQ: (h) mass spectrum of DMDS adduct of 411-12:Me, (i) mass spectrum of DMDS adduct of Z9-14:Me, (j) mass spectrum of DMDS adduct of E/Z11-14:Me, (k) mass spectrum of DMDS adduct of Z11-16:Me. (1-n) Quantification of saturated and unsaturated products made by the desaturase Ecau_Des4_ASVQ, Ecau_Des11_VPVQ, Ecau_Des14_LPVQ. The experiments are replicated three times, and the error bars represent standard error.

FIGS. 4A-4G shows the GC-MS analysis of FAMEs from yeast cell expressing Ecau_Des4, 11, 14. desaturase supplemented with Z9-14:Me*. GC chromatogram of FAME derived from (a) yeast cells transformed by Ecau_Des4_ASVQ, (b) yeast cells expressing Ecau_Des11_VPVQ, (c) yeast cells expressing Ecau_Des14_LPVQ. Yeast cells are supplemented with Z9-14:Me*. (d) Mass spectrum of Z9,E12-14:Me from Ecau_D4_ASVQ yeast sample. (e) Mass spectrum of DMDS derivative of Z9,E12-14:Me from Ecau_D4_ASVQ yeast sample. (f) the empty virus infected Sf9 cell fed with Z9-14:Me. (g) Ecau_D4_ASVQ_bacmid infected Sf9 cell fed with Z9-14:Me.

FIG. 5 shows the ZETA pheromone biosynthetic pathway in Ephestia cautella. Palmitate is (first) desaturated to Z11-16:Acyl, which is then chain shortened (β-oxidation) to Z9-14:Acyl. Z9-14:Acyl is (omega) desaturated to Z9,E12-14:Acyl, which is reduced and acetylated to the final ZETA pheromone. The genes characterized in this study are denoted in bold type.

FIG. 6 shows the reconstructed ZETA biosynthetic pathway in Saccharomyces cerevisiae. (a) biosynthetic pathway leading to the bioproduction of ZETA pheromone. (b) genetic circuit for the yeast transformation. (c) GC trace of heptane extract of the yeast media. (d) mass spectrum of the yeast-produced ZETA pheromone.

FIG. 7 shows the GC-EAD bioassay of yeast produced pheromone Z9,E12-14:OAc. Antennal responses (EAD) from male Plodia interpunctella and Spodoptera exigua elicited by GC elutes (FID) of synthetic compounds Z9-14:OAc and Z9,E12-14:OAc (a), ester fraction of yeast cell pellet extract (b), and crude extract of medium (c).

FIG. 8 shows coexpression of the DmeD9 and EcauD4 produced the doubly unsaturated product Z9E12-14:Acyl in N. benthamiana. TE14 was used to boost the amount of the precursor 14:CoA. DmeD9 introduced the first double bond in the acyl chain and Z9-14:CoA is produced, which is then used as a precursor for the EcauD4 to produce the doubly unsaturated product Z9E12-14:Acyl. This doubly unsaturated fatty acid can be further converted to Z9E12-14:OAc, the ZETA pheromone.

### DETAILED DESCRIPTION

The detailed description set forth below is intended as a description of the presently preferred embodiment of the invention, and is not intended to represent the only form in which the present invention may be constructed or utilized. The description sets forth the functions and sequences of steps for constructing and operating the invention. It is to be understood, however, that the same or equivalent functions and sequences may be accomplished by different embodiments and that they are also intended to be encompassed within the scope of the invention.

### METHODS AND MATERIALS

Identification of Pheromone Biosynthesis Candidate Desaturases from Ephestia cautella Model.

Insect rearing and tissue collection. The E. cautella individuals were reared on an artificial diet at 25±2.0° C. under an L16:D8 photoperiod as previously described in the field. Freshly emerged female adults were collected daily before the scotophase and considered to be 0 d old. The female pupae were collected separately, and the newly emerged adults were maintained in a vial under the same conditions. Laboratory cultures of S. exigua and P. interpunctella were reared at 23±1° C. under a 17:7 L:D photoperiod and 60% relative humidity and fed on wheat-germs based and beans based artificial diet, respectively. The male and female pupae were kept separately. Adults were fed with a 10% honey solution, and two- to three-day-old unmated males were used for the electrophysiological experiments.

Desaturase cDNA cloning. Among twenty-two desaturases transcripts reported in the E. cautella PG transcriptome, we chose six highly expressing candidates, viz., E. cautella desaturase 2 (abbreviated as Ecau_D2), Ecau_D4, Ecau_D6, Ecau_D9, Ecau_D11, and Ecau_D14 as the potential desaturases for the further yeast expression and assays. The full-length open reading frame (ORF) sequences of these desaturases were obtained by amplifying both the 5′ and 3′ cDNA ends using the rapid amplification of cDNA ends (RACE) technique (Table S1). The cDNAs were prepared from the E. cautella PG total RNA (approximately 1 μg) using the SMARTer RACE Kit (Clontech, Mountain View, CA, USA). The total RNA of E. cautella PGs (n=30; 1-4 days old female adults dissected at late scotophase) was prepared using a PureLink RNA Mini Kit (Thermo Fisher, Waltham, MA, USA). Touchdown polymerase chain reaction (PCR) [95° C. for 5 min, 35 cycles of 95° C. for 1 min, 65° C. (touchdown to 55° C.) for 30 s and 72° C. for 3 min; and one cycle at 72° C. for 10 min] was carried out using Advantage 2 PCR kit (Clontech), and the PCR products were gel-purified (Wizard SV Gel purification kit-Promega, Madison, WI, USA) cloned into the pGEM-T vector (Promega) followed by transformation into JM109 competent cells (Promega). The plasmids were isolated and sequenced in both directions (ABI 3500, Thermo Fisher) for sequence verification (see primer details in Table S1).

TABLE 1

| Primer name | Sequence |
|---|---|
| EP_Desat2_qRT_F | TTGCTTGAAGGAGCGAAGATTGG |
| EP_Desat2_qRT_R | AATGCGATGGTCCCTTACCCAGT |
| EP_Desat11_qRT_F | ATGCCTACGTTTGTGCCGGTCT |
| EP_Desat11_qRT_R | GCGGCGCTATTGACAAGGAAAG |
| EP_Desat14_qRT_F | CGATCATCGCTTGCACCACAGA |
| EP_Desat14_qRT_R | CCCACGAATGGAATGGCGTATT |
| EP_Desat4_qRT_F | CTTCCCATATGTTCGGCTACAA |
| EP_Desat4_qRT_R | GTTGTGGTAACCCTCTCCTAATG |
| EP_DesatZ9_qRT_F | TTTCCGTTTCCGTATTCGCTCTT |
| EP_DesatZ9_qRT_R | GGTCATACGCCCATCCGATTTT |
| EP_Desat6_qRT_F | TGCCGTTGACCTGTTTCATTCTG |
| EP_Desat6_qRT_R | GATGTTCTTGTCGTAAGGCTTGCTG |
| EP_Ef1_Refgene_F | CTGTCAACTTCAAGGATGCTACGA |
| EP_EF1_Refgene_R | GCCATTCTTTCCTTCTGAACACCT |
| EP_Act-F | TCCTACGAACTTCCCGACGGTCAAG |
| EP_Act-R | TACATGGTGGTACCTCCGGACAATA |
| EP_desat2_3′F1 | CTGGGTAAGGGACCATCGCATTC |
| EP_desat2_3′F2 | GACCATCGCATTCACCACAAGT |
| EP_desat2_5′R1 | CGATTACCCCATTTATGCCCGATG |
| EP_desat2_5′R2 | ATGCCCGATGCTGTTGACCAAG |
| EP_desat4_3′F1 | CAGCACCGCCTGGCATGTCAAT |
| EP_desat4_3′F2 | CGCCTGGCATGTCAATTTGCTG |
| EP_desat4_5′R1 | AAATTGACATGCCAGGCGGTGC |
| EP_desat4_5′R2 | TGCCAGGCGGTGCTGATACTCT |
| EP_desat6_3′F1 | GGGGCAGCAAGCCTTACGACAA |
| EP_desat6_3′F2 | AGCCTTACGACAAGAACATCAACCCC |
| EP_desat6_5′R1 | GCCTGGTTTCTACGGGGTTGATG |
| EP_desat6_5′R2 | TCTACGGGGTTGATGTTCTTGTCG |
| EP_D9_3RACE_F | ACATTGGCTGGCTGCTGGTTAGG |
| EP_D9_5RACE_R | CCAGCCCTTTGCCTTTCCTCTTA |
| EP_D11_3RACE_F | GAAGCACCCTGAAGTCAAAGCAA |
| EP_D11_5RACE_R | GCTTTGACTTCAGGGTGCTTCTT |
| EP_D14_3RACE_F | GGATCGGCTGGGCTTATGACTTGA |
| EP_D14_5RACE_R | TCAAGTCATAAGCCCAGCCGATCC |
| attB1_ephD2_F | GGGGACAAGTTTGTACAAAAAAGCAGGCTTCGACAA |
| | TGGAAAAGGACACTCAAAACGACGT |
| attB2_ephD2_R | GGGGACCACTTTGTACAAGAAAGCTGGGTCTAACCTTT |
| | ACAATATAACAGTTTTACATTC |
| attB1_ephD4_F | GGGGACAAGTTTGTACAAAAAAGCAGGCTATGACTCCC |
| | AACCCAGAATCTACTG |
| attB2_ephD4_R | GGGGACCACTTTGTACAAGAAAGCTGGGTTCAAAAATC |
| | ACTGTTTACTTCATTTTCA |
| attB1_ephD6_F | GGGGACAAGTTTGTACAAAAAAGCAGGCTACATGCCAC |
| | CTCAAGGTCAAGAGCGAG |
| attB2_ephD6_R | GGGGACCACTTTGTACAAGAAAGCTGGGTCTTATTCAC |
| | TTTTGGACGGGTTGATGA |
| attB1_ephD9_F | GGGGACAAGTTTGTACAAAAAAGCAGGCTTCGTGAACA |
| | TGGCTCCAAACGCTACAGAT |
| attB2_ephD9_R | GGGGACCACTTTGTACAAGAAAGCTGGGTCTTAGTCGTC |
| | TTTTGGGTGCAATCTAATAG |
| attB1_ephD11_F | GGGGACAAGTTTGTACAAAAAAGCAGGCTATGGTTCCGT |
| | ATAAGGATTCTAGTAC |
| attB2_ephD11_R | GGGGACCACTTTGTACAAGAAAGCTGGGTTCAAAAGTCA |
| | CTATAGGTTTCATTAGAATTATC |
| attB1_ephD14_F | GGGGACAAGTTTGTACAAAAAAGCAGGCTATGGTTCCGT |
| | ATAAGGATTCCGATG |
| attB2_ephD14_R | GGGGACCACTTTGTACAAGAAAGCTGGGTTTAATTATCC |
| | AAGTCATTATTGGTTTCAGT |
| SmartRACE_UPM | CTAATACGACTCACTATAGGGCAAGCAGTGGTATCAA |
| | CGCAGAGT |
| M13 Forward | CGCCAGGGTTTTCCCAGTCACGAC |
| M13 Reverse | TCACACAGGAAACAGCTATGAC |
| SmartRACE_NUP | AAGCAGTGGTATCAACGCAGAGT |

Quantitative desaturase expression analysis. RT-qPCR assessed the relative expression of six desaturases in the PG. E. cautella PG cDNAs were prepared from RNA (˜ 1 μg) of different age groups (1, 2, 4, 6, and 10 days old), using a PureLink RNA Mini Kit (Thermo Fisher). According to the manufacturer's instructions, the first-strand cDNA was synthesized using SuperScript IV Reverse Transcriptase (Invitrogen, Carlsbad, CA, USA). RT-qPCR assays were performed in the Applied Biosystems® 7500 Fast Real-Time PCR Systems (SYBR Green PCR Master Mix, Thermo Fisher). Desaturase expression was calculated from three biological replicates obtained from 20 E. cautella female PG dissected during late scotophase. 25 μL technical triplicates were run on the Applied Biosystems® 7500 Fast Real-Time PCR Systems using 100 ng PG cDNA template and 100 mM gene-specific primers (see primer details in Table S1) using the thermal program: 50° C. for 20 see (precycling); 95° C. for 5 min (holding); 40 cycles of 95° C. for 15 s and 55° C. for 32 s; and finally a continuous melting curve step of 95° C. for 15 s, 60° C. for 1 min, 95° C. for 30 s, and 60° C. for 15 s. Relative expression levels of different desaturase genes were measured with the 2-ACT method by normalizing them to housekeeping genes, actin, and elongation factor (endogenous control) (Table S1). The qPCR data were analyzed using the software in the 7500 Fast Real-Time PCR System, and significant change in desaturase expression was estimated by one-way analysis of variance (ANOVA), followed by multiple-comparison with the least significant difference (LSD) test (P<0.05) using SPSS v.24 (IBM SPSS statistics, NY, USA). Desaturase phylogenetic analysis. We aligned amino acid sequences using MAFFT. We used the auto algorithm and BLOSUM30 as the scoring matrix. The final multiple sequence alignment contained 122 sequences with 541 amino acid sites. The phylogenetic tree was constructed in IQ-TREE/2.0-rc2 (http://www.iqtrec.org; last accessed: Apr. 21, 2021). The automatic model search was performed using ModelFinder. The maximum likelihood analysis was performed using default settings and ultrafast bootstrap support with 5000 replicates. We used the Geneious (version 9.1, created by Biomatters, available from http://www.gencious.com/) for alignment construction, visualizing, and annotating the phylogenetic tree. The terminology for desaturases coined based on the widely used ‘signature motif’ within a supported grouping of lepidopteran sequences was used when appropriate.

### Heterologous Expression.

Functional expression of key PG desaturases-Yeast. For constructing a yeast expression vector containing a candidate desaturase gene, specific primers (Table S1) with attB1 and attB2 sites incorporated were designed to amplify the ORF. The PCR products were subjected to agarose gel electrophoresis and purified using the Wizard® SV Gel and PCR Clean-up system (Promega). The ORF was cloned into the pDONR221 vector in the presence of BP clonase (Invitrogen); after confirmation by sequencing, the correct entry clones were selected and subcloned to the pYEX_CHT_DEST vector, and recombinant constructs were analyzed by sequencing again. The final recombinant expression clones harboring the E. cautella desaturase genes were introduced into the olellelol disruption strain of the yeast Saccharomyces cerevisiae using the S.c. easy yeast transformation kit (Invitrogen). For the selection of uracil and leucine prototrophs, the transformed yeast was allowed to grow on an SC-U plate containing 0.7% YNB (w/o aa, Sigma, St. Louis, MO, USA) and a complete drop-out medium lacking uracil and leucine (Formedium, Norfolk, UK), 2% glucose, 1% tergitol (type Nonidet NP-40, Sigma), 0.01% adenine (Sigma) and containing 0.25 mM oleic acid (Sigma) as an additional fatty acid source. After five days at 30° C., individual colonies were picked up to inoculate 1 mL selective medium (SC-U), which was grown at 30° C. at 300 rpm for 48 h. Yeast cultures were diluted to an OD600 (measured by Pharmacia Ultrospec 3000, Cambridge, UK) of 0.4 in 5 mL fresh selective medium containing 1 mM CuSO4 with supplementation of a biosynthetic precursor. Each fatty acid methyl ester (FAME) precursor [Lauric acid methyl ester, 12:Me; myristic acid methyl ester, 14:Me; (Z)-9-tetradecenoic acid methyl ester, Z9-14:Me] was prepared in a concentration of 100 mM in 96% ethanol and added to reach a final concentration of 0.5 mM in the culture medium. Compound acronyms refer to geometry across the double bond, position of unsaturation, carbon-chain length, and functionality; e.g., Z9-14:Me=(Z)-9-tetradecenoic acid methyl ester. Me=methyl ester, OH=fatty alcohol, OAc=alcohol acetate ester. We used FAME as supplemented precursors because they are more soluble in the medium than free fatty acids. Yeasts were cultured at 30° C. 300 rpm. After 48 h, yeast cells were harvested by centrifugation at 4,000 g, and the medium was discarded. Experiments were repeated three times. The pellets were stored at −80° C. until fatty acid analysis. Heterologous Expression of Desaturases in Insect Cells. To further confirm the function of Ecau_D4, we expressed this desaturase in the Spodoptera frugiperda Sf9 cells using the Baculovirus expression system. The expression construct for Ecau_D4 in the baculovirus expression vector system (BEVS) donor vector pDEST8_Ecau_D4 was made by LR reaction. Recombinant bacmids were made according to instructions for the Bac-to-Bac® Baculovirus expression system given by the manufacturer (Invitrogen) using DH10EMBac Y (Geneva Biotech, Genève, Switzerland). Baculovirus generation was done using S. frugiperda Sf9 cells (Thermo Fisher Scientific), Ex-Cell 420 serum-free medium (Sigma), and baculoFECTIN II (OET, Oxford, UK). The virus was then amplified once to generate a P2 virus stock using Sf9 cells and Ex-Cell 420 medium. Viral titer in the P2 stock was determined using the BaculoQUANT all-in-one qPCR kit (OET) and found to be: 0.8×108 pfu/mL for Ecau_D4_ASVQ. Insect cell lines, Sf9 were diluted to 2×10⁶ cells/mL. Heterologous expression was performed in 20-ml cultures in Ex-Cell 420 medium, and the cells were infected at an MOI of 1. The cultures were incubated in 125 ml Erlenmeyer flasks (100 rpm, 27° C.), with fatty-acid methyl-ester substrates supplemented at a final concentration of 0.25 mM after one day. After three days, 7.5-mL samples were taken from the culture and centrifuged for 15 min at 4500 g at 4° C. The pellets were stored at −80° C. until fatty acid analysis. Sf9 expression experiments were conducted in three replicates.

Fatty acid analysis of yeast and Sf9 cells. Total lipids were extracted from yeast cells using 1 mL of methanol/chloroform (2:1, v/v, containing 3.12 μg/mL of 19:Me as internal standard) in a glass tube. One mL of HAc (0.075 M) was added to each tube and then vortexed vigorously. Phase separation was achieved by centrifuging at 2,000 rpm for 2 min. The bottom chloroform phase, about 0.33 mL containing the total lipids, was transferred to a new glass tube. FAMEs were made from this total lipid extract. The solvent was evaporated to dryness under gentle nitrogen flow. One mL of sulfuric acid 2% (w/w) in methanol was added to the tube, vortexed vigorously, and incubated at 90° C. for one hour. After incubation, 1 mL of water was added, mixed well, and then 1 mL of heptane was used to extract the FAMEs. The methyl ester samples were subjected to GC-MS analysis on an Agilent 8890 GC equipped with an INNOWax column (30 m×0.25 mm i.d., 0.25 μm film thickness) coupled to a mass detector Agilent 5977B. GC thermo program was set to be held at 80° C. for 1 min, then ramped up to 230° C. at a rate of 10° C. min-1 and held for 15 min followed by a post-run at 240° C. for 3 min. MS scan range was m/z 30 to 350.

Double bond positions of monounsaturated compounds were confirmed by dimethyl disulfide (DMDS) derivatization followed by GC-MS analysis. DMDS reaction was performed by adding 50 μL of DMDS (Sigma) to 50-100 μL of FAME (biological samples) and in the presence of 5 μL of iodine (5% in DEE) as a catalyst. Reactions were incubated at 40° C. overnight. In the case of identifying diene in the biological samples, incubation was done at 55° C. for 48 hours. After that, 50 μL of Na₂S₂O₃ solution (5% in water) was used to neutralize the iodine, and the organic phase was transferred to a new tube and condensed to about 40 μL. GC-MS analysis of DMDS product was done in Agilent 7890A equipped with HP-5 column (30 m×0.25 mm i.d., 0.25 μm film thickness) coupled to a mass detector Agilent 5975C. GC thermo program was set at 80° C. for 1 min, raised to 140° C. at a rate of 20° C./min, then to 250° C. at a rate of 4° C./min and held for 20 min.

### Establishment of ZETA Pathway in Yeast System for Bioproduction.

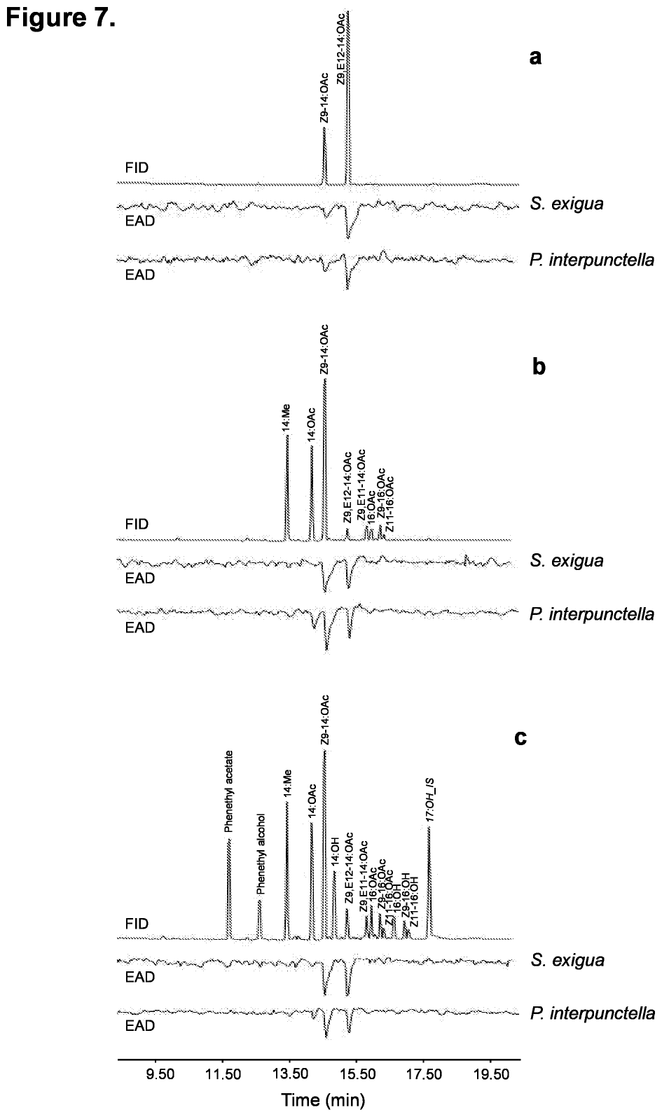

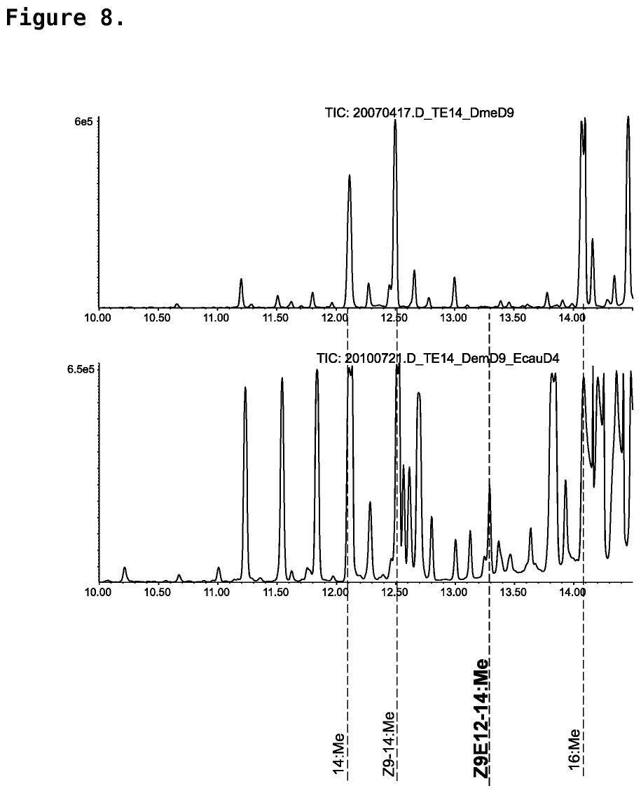

Assembly of biosynthetic pathway in yeast and bioproduction of ZETA. Desaturase Dmel_D9 from Drosophila melanogaster, Ecau_Des4_ASVQ characterized in this study, fatty acyl reductase SexipgFARII from Spodoptera exigua, and acetyltransferase ATF1 from Saccharomyces cerevisiae were synthesized (by GeneArt strings, Thermo Fisher and codon-optimized for S. cerevisiae), assembled by fusion PCR and Gateway assembly (Thermo Fisher), cloned to the expression vector pYEX_CHT_DEST (FIG. 7B). Yeast strain INVSc1 (Invitrogen) was transformed as described above. Single colonies were inoculated in a shaking flask with 100 mL SC-U media and cultivated at 30° C. with Galactose (2%, final concentration) and Cu²⁺ (1 mM) as an induction agent. Myristic acid methyl ester, final concentration 0.5 mM) is supplemented as the starting material for the sex pheromone biosynthetic pathway. After four days of growth, cells are collected by centrifugation (4,000 g, 10 min). We used heptane to extract 25 mL of the medium and condensed the heptane extract to 1 mL, and then 2 μL was injected for GC analysis using the protocols above.

Electrophysiology of ZETA yeast produced pheromone. The antennal electrophysiological activity of the yeast-derived pheromone was tested by gas chromatography with electroantennographic detection (GC-EAD). An Agilent 7890 gas chromatograph equipped with a flame ionization detector (FID), an HP-INNOWax column (30 m×0.25 mm i.d., and 0.25 μm film thickness; J&W Scientific, USA) was used. Antennae of male S. exigua and P. interpunctella with both tips cut-off and associated with the head were mounted on a PRG-2 EAG (10× gain) probe (Syntech, Kirchzarten, Germany) using conductive gel (Blågel, Cefar, Malmö, Sweden). The antennal preparation was put in a flow of charcoal-filtered and humidified air that passed through the column outlet. Hydrogen was used as the carrier gas with a constant flow of 1.8 mL/min, and the GC effluent was directed to the FID and EAD by a 1:1 division. The GC inlet was set at 250° C., the transfer line was set at 255° C., and the detector was set at 280° C. The GC oven was programmed from 80° C. for 1 min, then increased to 210° C. at a rate of 10° C./min and held for 10 min. Data were collected with the software GC-EAD Pro Version 4.1 (Syntech).

### Results and Discussion

Identification of Pheromone Biosynthesis Candidate Desaturases from Ephestia cautella.

Desaturase cDNA cloning. The ORF cDNA transcripts of Ecau_D2, Ecau_D4, Ecau_D6, Ecau_D9, Ecau_D11, and Ecau_D14 cDNA contain 993 bp (SEQ ID: 1), 1017 bp (SEQ ID: 2), 1059 bp (SEQ ID: 3), 1059 bp (SEQ ID: 4), 1029 bp (SEQ ID: 5), and 1035 bp (SEQ ID: 6), respectively, and correspond to proteins of 330 aa (Ecau_D2) (SEQ ID: 7), 338 aa (Ecau_D4) (SEQ ID: 8), 352 aa (Ecau_D6) (SEQ ID: 9), 352 aa (Ecau_D9) (SEQ ID: 10), 342 aa (Ecau_D11) (SEQ ID: 11) and 344 aa (Ecau_D14) (SEQ ID: 12) with predicted molecular weights of 38.63, 39.42, 40.31, 40.70, 39.81 and 39.90 kDa, respectively (GenBank acc nos. MW922324 to MW922329). Ecau_D2 shares 93.27% identity with the Cydia pomonella delta-(9)-fatty acyl desaturase (GenBank acc no. AIM40219.1). Ecau_D6 and Ecau_D9 share 89.52% and 94.35% identity, respectively, with the Amyelois transitella acyl-CoA delta-(9)-desaturases (EC 1.14.19.2). Whereas, Ecau_D4, Ecau_D11, and Ecau_D14 are the acyl-CoA delta-(11)-desaturase (EC 1.14.19.5) shares 73.61%, 82.35%, and 80.49% identity respectively with A. transitella (GenBank acc no. NP_001299594.1). The endoplasmic reticulum localization of all these desaturases, a typical characteristic of moth desaturase proteins, was identified using the Euk-mPloc 2.0 server.

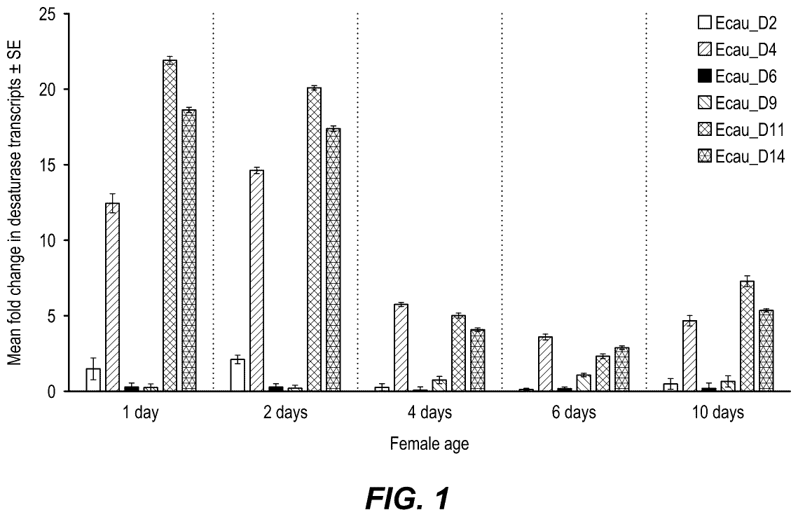

Relative expression level of E. cautella desaturses. Identifying the PG-enriched expression of the desaturase gene can provide valuable insights into its role in pheromonogenesis. We quantitatively measured the expression of all the six candidates using qRT-PCR, and the rate of the desaturase expression between different ages (1-10 days) was compared. We observed a significantly higher expression level (P<0.001) of Ecau_D11 and a slightly lower expression of Ecau_D14 in the 1-2 days old female E. cautella. When compared to Ecau_D2 expression in 1-day E. cautella, we found fourteen-times higher expression in Ecau_D11 (F=2106; dƒ=1, 10; P<0.0001), followed by Ecau_D14 (F=2041; dƒ=1, 10; P<0.0001), with twelve-times higher expression, as shown in FIG. 1. Our data also showed a higher expression of Ecau_D4 in 1-day old females, which is approximately eight times higher than Ecau_D2 (F=547; dƒ=1, 10; P<0.0001), as shown in FIG. 1. Ecau_D11 was slightly down-regulated in the 2-day old females compared to 1-day old insects. In contrast, up-regulation of Ecau_D4 was observed in the same stage. However, the mRNA expression level of all these three genes was much lower (in 4-10 days old females, as shown in FIG. 1. In contrast, the other three desaturases, Ecau_D2, Ecau_D6, and Ecau_D9, were found to be poorly expressed in the E. cautella PG.

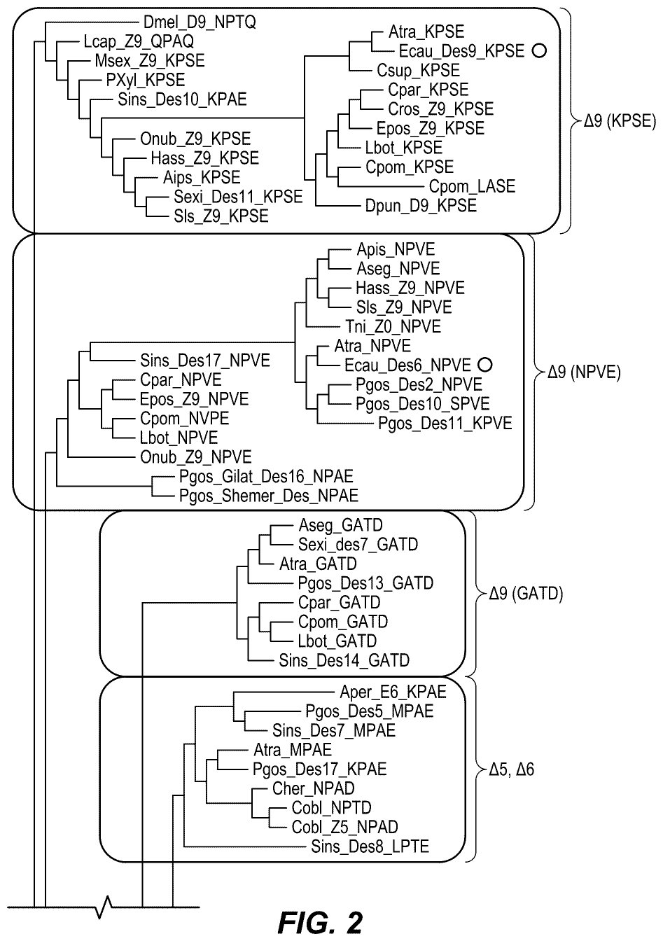

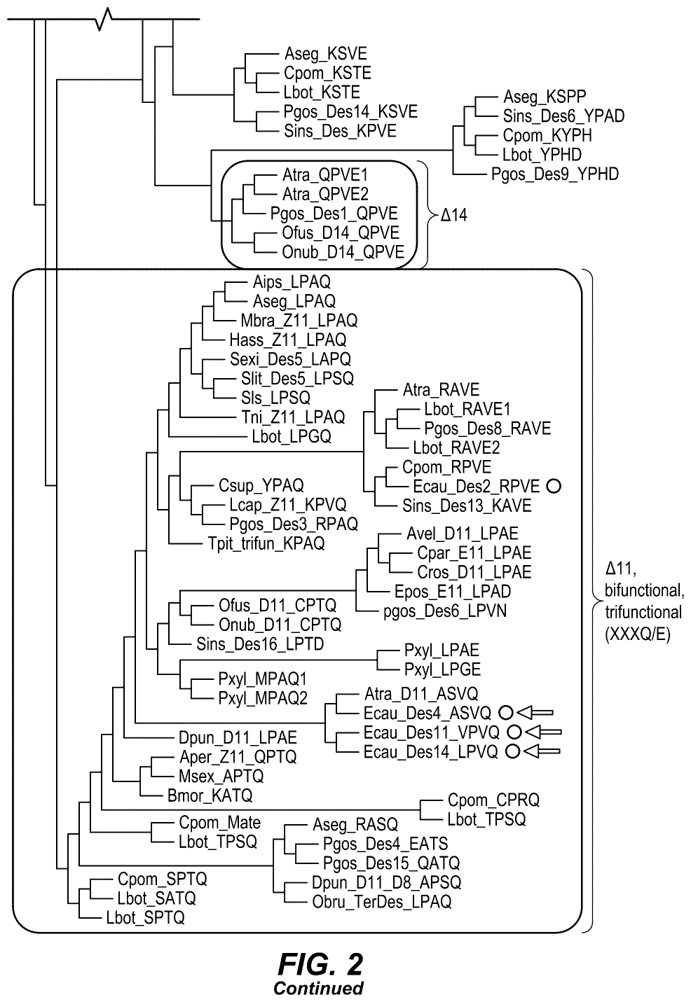

Desaturase phylogenetic analysis. Desaturase sequences obtained from transcriptome and genome projects of various moth species were selected for building the phylogenetic tree, as shown in FIG. 2. Our sampling of desaturase sequences includes the functional classes previously identified in moths (i.e., Δ5, Δ6, Δ8, Δ9, Δ10, Δ11, Δ12, Δ14). The results revealed that Ecau_Des6_NPVE and Ecau_Des9_KPSE are categorized as metabolic desaturase essential for lipid metabolism, cell signaling, maintaining membrane fluidity in response to temperature fluctuation. Most likely, these desaturases are not involved in pheromone biosynthesis. Ecau_Des4_ASVQ, Ecau_Des11_VPVQ, and Ecau_Des14_LPVQ fall into the A11 desaturase clade with signature motif XXXQ/E, the most relevant clade of desaturases to moth sex pheromone biosynthesis. This group contains many genes involved in the biosynthesis of diene pheromone, e.g., Atra_ASVQ in Amyelois transitella, Bmor_KATQ in Bombyx mori, Msex_APTQ in Manduca sexta, Lcap_KPVQ in Lampronia capitella, Dpun_LPAE in Dendrolimus punctatus, Sexi_LPAQ, Slit_LPAQ, and Sls_LPSQ in Spodoptera spp, and Cpo_CPRQ in Cydia pomonella. Ecau_Des2_RPVE falls into the same desaturase clade and is close to Lcap_KPVQ, which has proven to desaturate Z9-14:Acyl to Z9,Z11-14:Acyl, the immediate fatty acyl precursor of the Lampronia capitella pheromone.

### Heterologous Expression.

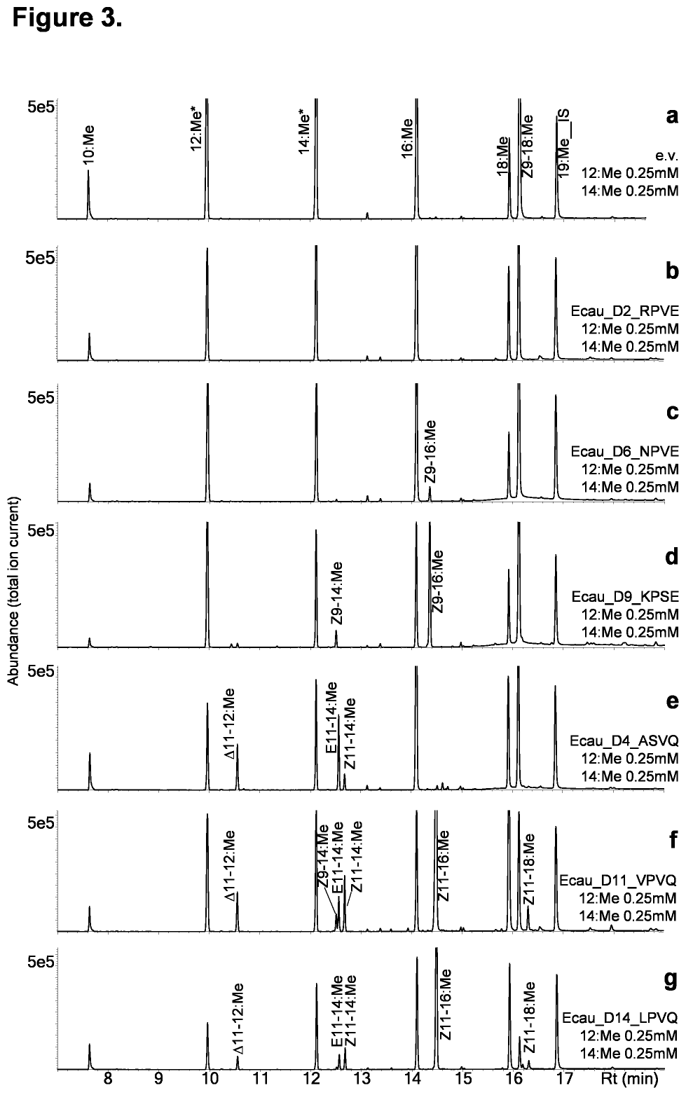

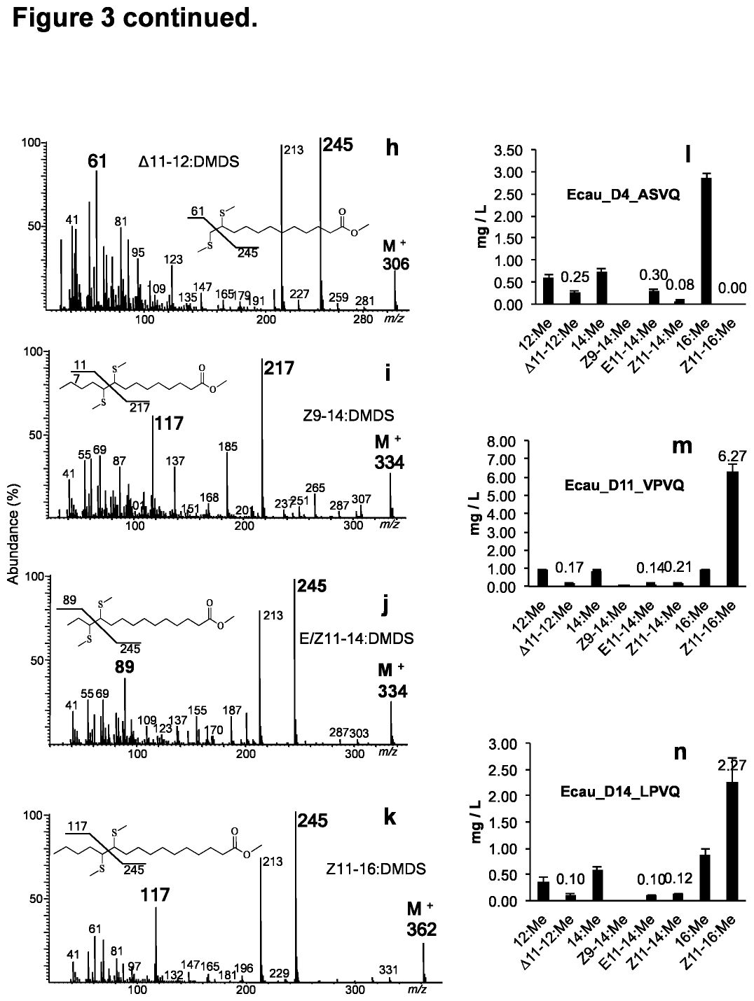

Functional expression of candidate PG desaturases in the Yeast and Sf9 cell. We expressed all the six desaturases in our yeast expression system. In the first round of experiments, the yeast was fed with 12:Me and 14:Me [yeast cells have enough C16 (palmitic acid) and C18 (stearic acid) for desaturase to use as substrate] to get an overview of the activity of each desaturase. As shown in FIG. 3a-g, Ecau_D6_NPVE and Ecau_D9_KPSE are metabolic desaturases producing Z9-14:Acyl, (Z)-9-hexadecenoic acid (Z9-16:Acyl), and (Z)-9-octadecenoic acid (Z9-18:Acyl). Ecau_D6_NPVE and Ecau_D9_KPSE are categorized as metabolic desaturases. Most likely, these desaturases are not involved in pheromone biosynthesis, with a few exceptions reported in previous studies. For example, the Dpu_KPSE from Dendrolimus punctatus (Lepidoptera: Lasiocampidae) produces a range of 49-monounsaturated products. When supplemented with Z7- and E7-14:Acyl [(Z)-7-tetradecenoic acid and (E)-7-tetradecenoic acid], Dpu_KPSE can introduce a second desaturation to produce Δ7Δ9-14:Acyl, illustrating the formation of conjugated double bonds by this type of fatty acyl desaturase. In our yeast assay, Ecau_D9_KPSE produced a small amount of Z9-14:Acyl, which can be used as a substrate for the Ecau_D4_ASVQ to produce the doubly unsaturated product Z9,E12-14:Me. Maybe Ecau_D9_KPSE can also contribute to the pheromone biosynthesis in the live insect. When Ecau_Des4_ASVQ is expressed in yeast, it produces more of the E isomer than the Z isomer of the monounsaturated Δ11-14C products. Moreover, this desaturase showed no activity on C16 in yeast, as seen in FIG. 3c. On the contrary, the other two closely related desaturases show high activity on C16, as seen in FIG. 3f-g. Ecau_D11_VPVQ and Ecau_D14_LPVQ mainly produce (Z)-11-hexadecenoic acid Me, Z11-16:Me, which is a precursor for pheromone production. These desaturases were also found to act on C14:Acyl, making small amounts of (E)-11-tetradecenoic acid (E11-14:Acyl) and (Z)-11-tetradecenoic acid (Z11-14:Acyl). The Z9-14:Acyl in the Ecau_Des11_VPVQ yeast sample could be the chain-shortening product from the Z11-16:Acyl since this desaturase produces a large amount of Z11-16:Acyl. The DMDS reaction confirmed the double bond positions of all the unsaturated products, as seen in FIG. 3h-k. The relative quantity of desaturated product was calculated to understand the enzymatic activities on different carbon chain length substrates, as shown in FIGS. 31-n. Ecau_Des2_RPVE did not show any activity in our yeast expression system, which is expected since all its immediate neighbors are also non-functional desaturases, as can be seen in FIG. 2.

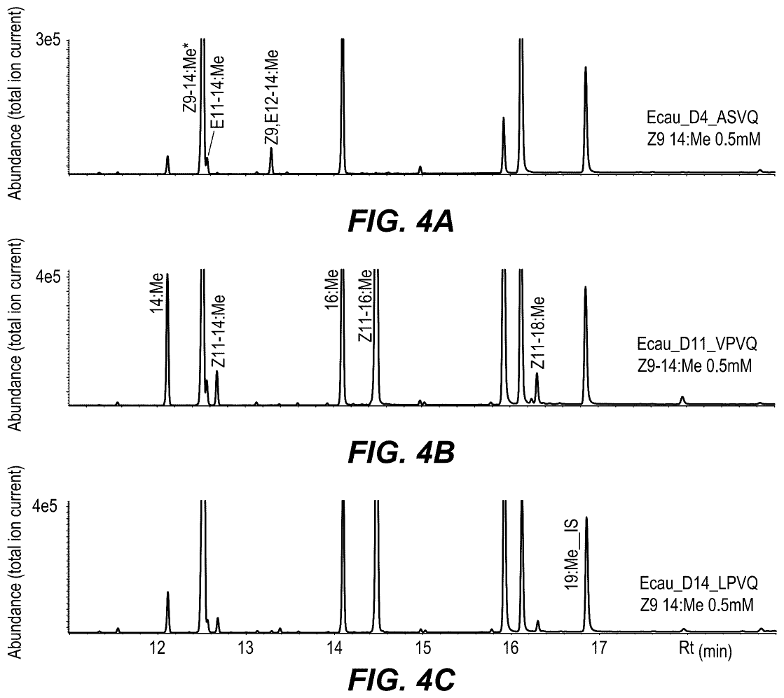

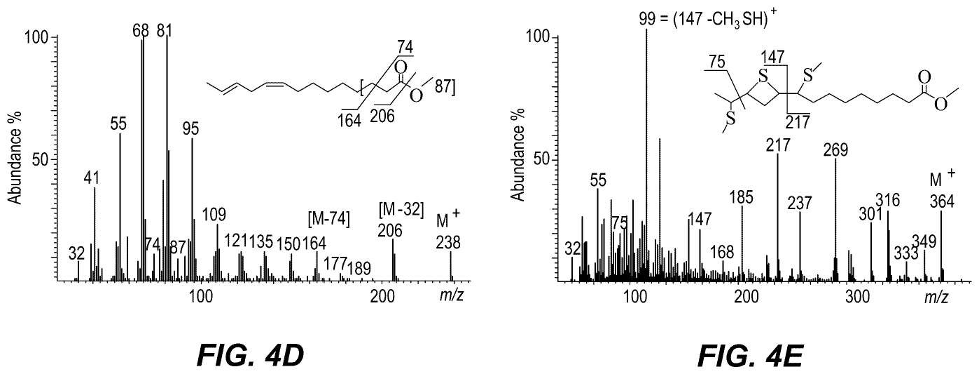

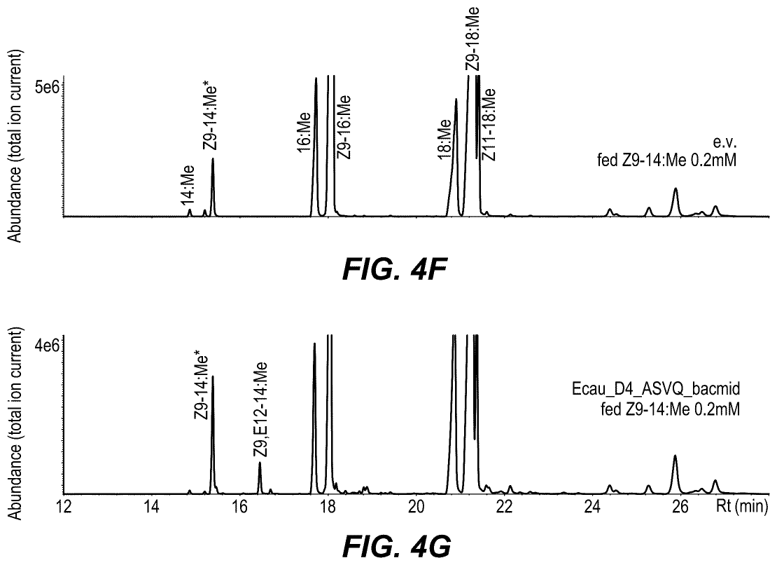

In the second round of the experiments, the three desaturases Ecau_Des4_ASVQ, Ecau_Des11_VPVQ, and Ecau_Des14_LPVQ were fed with Z9-14:Me to investigate if they could produce the diene pheromone precursor. As shown in FIG. 4A, Ecau_Des4_ASVQ can convert the Z9-14:Acyl into the (Z,E)-9,12-tetradecadienoic acid (Z9,E12-14:Acyl), a direct precursor for the subsequent reduction and acetylation steps. The mass spectrum of the Z9, E12-14:Me has m/z 68 and 81 as base peaks, supporting diene hydrocarbon moiety. Molecular ion m/z 238 has a relatively low abundance, typical for a diene with isolated double bonds, as shown in FIG. 4D. The DMDS results confirmed the positions of the double bonds to be 49 and A12, as shown in FIG. 4E. The other two desaturases, Ecau_D11_VPVQ, and Ecau_D14_LPVQ, did not produce the Z9,E12-14:Acyl, as shown in FIGS. 4B-C; hence they are not likely to be involved in the second desaturation step in the pheromone biosynthesis.

We further confirmed the activity of Ecau_Des4_ASVQ by expressing it in the insect cell line Sf9. Even though the catalytic activity of Ecau_Des4_ASVQ is reasonably good in the yeast, we were curious to see if the insect cells could provide a better cellular environment. When the Ecau_Des4_ASVQ was expressed in the Sf9 cell system and supplemented the culture medium with Z9-14:Me we observed a consistent production of Z9, E12-14:Acyl, demonstrating that Ecau_Des4_ASVQ indeed catalyzes the biosynthesis of Z9, E12-14:Acyl, as shown in FIGS. 4F-G. We did not detect any activity of this desaturase on longer chain length (C16, C18) substrates in Sf9 cells, which is in line with the previous results in yeast. The mass spectrum of the Z9,E12-14:Me peak was identical to the spectrum obtained from the yeast product.

### Establishment of ZETA Pathway in Yeast System for Bioproduction.

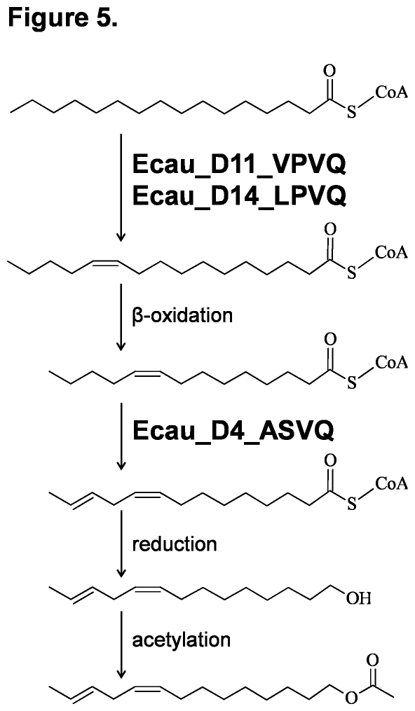

The proposed biosynthesis of Z9, E12-14:OAc in E. cautella, as recited in FIG. 5, is based on earlier in vivo labeling experiments. We conclude that Ecau_Des11_VPVQ and Ecau_Des14_LPVQ may contribute to the substrate pool of Z11-16: Acyl, which is then chain-shortened to Z9-14:Acyl subsequently serving as a substrate for Ecau_Des4_ASVQ to produce Z9,E12-14:Acyl, as shown in FIG. 5. This biosynthetic pathway involves at least two distinct desaturases, which is different from the previous study of a single bifunctional desaturase being responsible for producing monoene and diene products (with chain shortening in between) in Spodoptera spp.

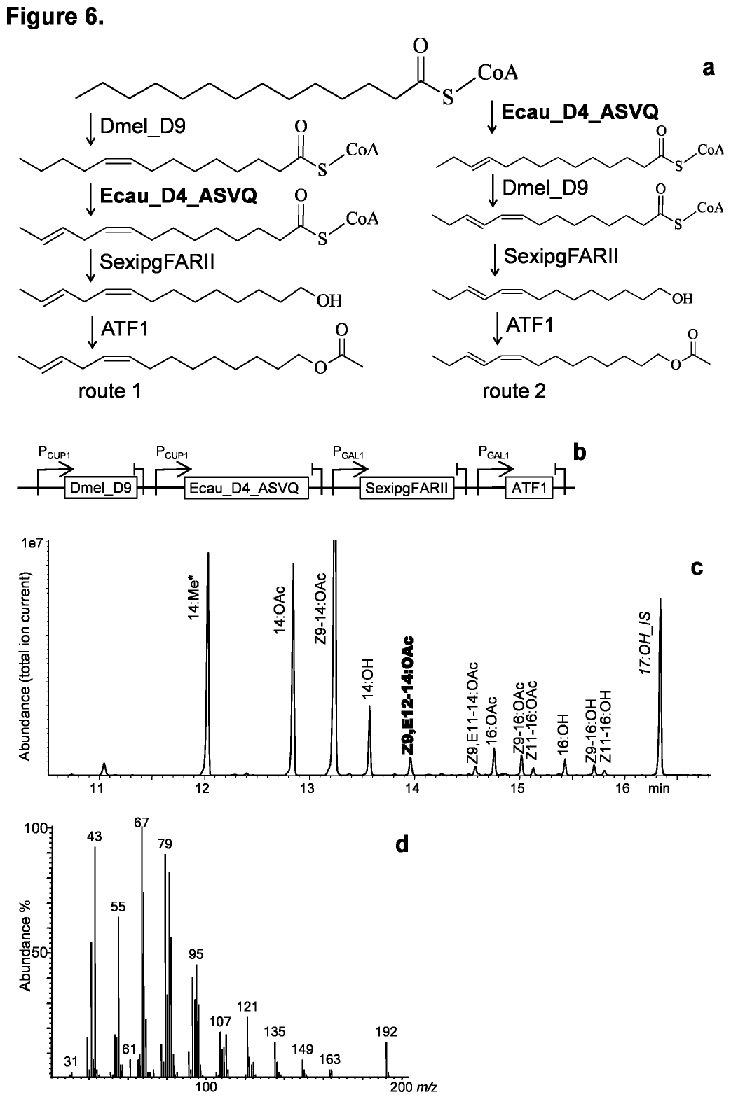

In order to construct the biosynthetic pathway for ZETA bioproduction, we used Dmel_D9 from D. melanogaster, which mainly produces Z9-14:Acyl when expressed in yeast. We do not have access to the PG-specific fatty acyl reductase (pgFAR) from E. cautella, and there has not been an insect-derived acetyltransferase characterized so far. Therefore, we used SexipgFARII and yeast ATF1 to assemble the biosynthetic pathway. The SexipgFARII is specialized for reducing the Z9,E12-14:Acyl, as previously demonstrated. The ATF1 is very active acetylating (Z, E)-9,12-tetradecadien-1-ol (Z9,E12-14:OH). When the entire biosynthetic pathway, as shown in FIGS. 6a-b. was expressed in yeast and supplemented with 14:Me in the yeast medium, the final pheromone component Z9,E12-14:OAc and (Z)-9-tetradecenyl acetate (Z9-14:OAc) were detected in the medium, as shown in FIGS. 6c-d, after four days of cultivation.

Interestingly, the acetylation step is very efficient, leaving no detectable level of intermediate Z9,E12-14:OH in the medium or yeast cells. Unfortunately, the activity of the Ecau_Des4_ASVQ is low in the yeast cellular environment, and thus this becomes a rate-limiting step in the biosynthetic pathway. Besides producing the ZETA pheromone, (Z,E)-9,11-tetradecadienyl acetate (Z9,E11-14:OAc, identity confirmed by comparing retention time with synthetic standard) was also produced in substantial amounts in the yeast strain (INVSc1), expressing the biosynthetic pathway. This may be caused by the different order of actions of the two desaturases, as can be seen in FIG. 6a. In route 1, Z9-14:Acyl is produced by Dmel_D9 first, then used as a substrate for the Ecau_Des4_ASVQ, followed by reduction and acetylation to make ZETA pheromone. In route 2, Ecau_D4_ASVQ uses 14:Acyl first to produce E11-14:Acyl, then Dmel_D9 uses the E11-14:Acyl as a substrate to produce Z9, E11-14:Acyl, this will lead to the production of Z9,E11-14:OAc. One way of suppressing route 2 would be to use a more potent promoter for the Dmel_D9. Hence, the Z9-14: Acyl would accumulate in the cells to provide substrate for the Ecau_D4_ASVQ, which under an inducible promoter could be activated after Z9-14:Acyl has reached a certain level. Along the same lines, the reductase and the acetyltransferase expression could be turned on at a later stage through the onset of the Gall promoter to avoid competing with the second desaturation step for the use of the Z9-14:Acyl pool. We calculated that the production titer in this study is 0.32 mg/L.

Electrophysiology activity of yeast-produced ZETA pheromone. The acetate products found in extracts of both yeast cell pellet and medium elicited clear responses from antennae of male P. interpunctella and S. exigua, as shown in FIG. 7. Both species responded strongly to ZETA and the monounsaturated Z9-14:OAc. The latter was produced in a much higher amount in the yeast compared to ZETA production. A minor response to the saturated tetradecanyl acetate was also observed from male P. interpunctella, whereas both species showed no response to other yeast-derived components.

To summarize, we successfully demonstrated the biological production of (Z,E)-9,12-tetradecadienyl acetate, the fatty acid-derived diunsaturated sex pheromone in yeast cell factories. Noticeably, the yeast produced ZETA pheromone in its correct isomeric form. Following confirmation of the chemical structure of the ZETA pheromone, electrophysiological studies proved ZETA pheromone induced typical antennal responses in male moths. The behavioral activity of the yeast-produced pheromone and the need for downstream processing of the raw product remains to be investigated. The practical application of bioproduction of ZETA pheromone is that the three enzymes used in the current study can effectively deliver the pheromone component. It is important to note that the yeast fermentation leftovers and byproducts in the present study are primarily biodegradable, and we used a standard medium in the experiment. In contrast, the chemical synthesis of ZETA will typically require unique starting material, expensive catalysts, and several synthesis steps, and above all, it produces several environmental pollutants. Our studies lay the foundation for the bioproduction of ZETA pheromones to be used in pheromone-based pest control of key agricultural and stored product pests such as Spodoptera spp., P. interpuctella, E. cautella and E. kuehniella.

### REFERENCES

1. FAO. Insect damage: damage on post-harvest. http://www.fao.org/3/av013e/av013e.pdf (2013). (last accessed on 24 Jul. 2021).
2. Ahmed S S and Hashem M Y, Susceptibility of different life stages of Indian meal moth Plodia interpunctella (Hübner) and almond moth Ephestia cautella (Walker) (Lepidoptera: Pyralidae) to modified atmospheres enriched with carbon dioxide. Journal of stored products research 51: 49-55 (2012).
3. Jung J-M, Nam Y, Jung S and Lee W-H, Spatial analysis of changes in Plodia interpunctella (Lepidoptera: Pyralidae) distribution depending on diets. Journal of Stored Products Research 91: 101777 (2021).
4. UNEP, 2014 report of the methyl bromide technical options committee, 2014 Assessment https://ozone.unep.org/sites/default/files/2019-05/MBTOC-Assessment-Report-2014.docx (2014). (last accessed on 24 Jul. 2021).
5. El-Saeid M H and Al-Dosari S A, Monitoring of pesticide residues in Riyadh dates by SFE, MSE, SFC, and G C techniques. Arabian Journal of chemistry 3: 179-186 (2010).
6. Osman K, Al-Humaid A, Al-Rehiayani S and Al-Redhaiman K, Monitoring of pesticide residues in vegetables marketed in Al-Qassim region, Saudi Arabia. Ecotoxicology and Environmental Safety 73: 1433-1439 (2010).
7. Antony B, Soffan A, Jakše J, Alfaifi S, Sutanto K D, Aldosari S A, et al., Genes involved in sex pheromone biosynthesis of Ephestia cautella, an important food storage pest, are determined by transcriptome sequencing. BMC genomics 16: 1-27 (2015).
8. Heuskin S, Verheggen F J, Haubruge E, Wathelet J-P and Lognay G, The use of semiochemical slow-release devices in integrated pest management strategies. Base 2011).
9. Petkevicius K, Löfstedt C and Borodina I, Insect sex pheromone production in yeasts and plants. Current opinion in biotechnology 65: 259-267 (2020).
10. Sammani AMP, Dissanayaka DMSK, Wijayaratne L K W and Morrison III W R, Effect of pheromone blend components, sex ratio, and population size on the mating of Cadra cautella (Lepidoptera: Pyralidae). Journal of Insect Science 20: 30 (2020).
11. Guerrero A, Malo E A, Coll J and Quero C, Semiochemical and natural product-based approaches to control Spodoptera spp.(Lepidoptera: Noctuidae). Journal of Pest Science 87: 231-247 (2014).
12. Löfstedt C and Xia Y-H. Biological production of insect pheromones in cell and plant factories. In Insect pheromone biochemistry and molecular biology. Elsevier, pp. 89-121 (2021).
13. Nansen C, Phillips T W, Morton P K and Bonjour E L, Spatial analysis of pheromone-baited trap captures from controlled releases of male Indian meal moths. Environmental entomology 35: 516-523 (2006).
14. Jurenka R. Lepidoptera: Female sex pheromone biosynthesis and its hormonal regulation. In Insect Pheromone Biochemistry and Molecular Biology. Elsevier, pp. 13-88 (2021).
15. Löfstedt C, Wahlberg N and Millar J G. 4. Evolutionary Patterns of Pheromone Diversity in Lepidoptera. In Pheromone communication in moths. University of California Press, pp. 43-78 (2016).
16. Rizvi S A H, George J, Reddy G V, Zeng X and Guerrero A, Latest developments in insect sex pheromone research and its application in agricultural pest management. Insects 12: 484 (2021).
17. Roelofs W L and Rooney A P, Molecular genetics and evolution of pheromone biosynthesis in Lepidoptera. Proceedings of the National Academy of Sciences 100: 9179-9184 (2003).
18. Moto Ki, Yoshiga T, Yamamoto M, Takahashi S, Okano K, Ando T, et al., Pheromone gland-specific fatty-acyl reductase of the silkmoth, Bombyx mori. Proceedings of the National Academy of Sciences 100: 9156-9161 (2003).
19. Antony B, Fujii T, Moto Ki, Matsumoto S, Fukuzawa M, Nakano R, et al., Pheromone-gland-specific fatty-acyl reductase in the adzuki bean borer, Ostrinia scapulalis (Lepidoptera: Crambidae). Insect biochemistry and molecular biology 39: 90-95 (2009).
20. Holkenbrink C, Ding B-J, Wang H-L, Dam M I, Petkevicius K, Kildegaard K R, et al., Production of moth sex pheromones for pest control by yeast fermentation. Metabolic Engineering 62: 312-321 (2020).
21. Petkevicius K, Koutsoumpeli E, Betsi P C, Ding B J, Kildegaard K R, Jensen H, et al., Biotechnological production of the European corn borer sex pheromone in the yeast Yarrowia lipolytica. Biotechnology Journal 16: 2100004 (2021).
22. Brady U E and Ganyard Jr M C, Identification of a sex pheromone of the female beet armyworm, Spodoptera exigua. Annals of the Entomological Society of America 65: 898-899 (1972).
23. Kuwahara Y, Kitamura C, Takashi S, Hara H, Ishii S and Fukami H, Sex pheromone of the almond moth and the Indian meal moth: cis-9, trans-12-tetradecadienyl acetate. Science 171: 801-802 (1971).
24. Hinks C and Byers J, Biosystematics of the genus Euxoa (Lepidoptera: Noctuidae): V. Rearing procedures, and life cycles of 36 species. The Canadian Entomologist 108: 1345-1357 (1976).
25. Heydorn P, Anderbrant O, Jonsson L J and Svensson G P, Long-term rearing affects pheromone-mediated flight behaviour of the Indian meal moth, Plodia interpunctella. Journal of Applied Entomology 143: 1193-1195 (2019).
26. Schmittgen T D and Livak K J, Analyzing real-time PCR data by the comparative C T method. Nature protocols 3: 1101-1108 (2008).
27. Katoh K, Rozewicki J and Yamada K D, MAFFT online service: multiple sequence alignment, interactive sequence choice and visualization. Briefings in bioinformatics 2017).
28. Minh B Q, Schmidt H A, Chernomor O, Schrempf D, Woodhams M D, Von Haeseler A, et al., IQ-TREE 2: new models and efficient methods for phylogenetic inference in the genomic era. Molecular biology and evolution 37: 1530-1534 (2020).
29. Kalyaanamoorthy S, Minh B Q, Wong T K, Von Haeseler A and Jermiin L S, ModelFinder: fast model selection for accurate phylogenetic estimates. Nature methods 14: 587-589 (2017).
30. Hoang D T, Chernomor O, Von Haeseler A, Minh B Q and Vinh L S, UFBoot2: improving the ultrafast bootstrap approximation. Molecular biology and evolution 35: 518-522 (2018).
31. Knipple D C, Rosenfield C-L, Nielsen R, You K M and Jeong S E, Evolution of the integral membrane desaturase gene family in moths and flies. Genetics 162: 1737-1752 (2002).
32. Patel O, Fernley R and Macreadie I, Saccharomyces cerevisiae expression vectors with thrombin-cleavable N- and C-terminal 6×(His) tags. Biotechnology letters 25: 331-334 (2003).
33. Schneiter R, Tatzer V, Gogg G, Leitner E and Kohlwein S D, Elolp-dependent carboxy-terminal elongation of C14:1 Delta (9) to C16:1 Delta (11) fatty acids in Saccharomyces cerevisiae. J Bacteriol 182: 3655-3660 (2000).
34. Ding B-J, Lienard M A, Wang H-L, Zhao C—H and Löfstedt C, Terminal fatty-acyl-CoA desaturase involved in sex pheromone biosynthesis in the winter moth (Operophtera brumata). Insect biochemistry and molecular biology 41: 715-722 (2011).
35. Ding B-J and Löfstedt C, Analysis of the Agrotis segetum pheromone gland transcriptome in the light of sex pheromone biosynthesis. BMC genomics 16: 1-21 (2015).
36. Ding B-J, Xia Y-H, Wang H-L, Andersson F, Hedenström E, Gross J, et al., Biosynthesis of the sex pheromonecomponent (E,Z)-7, 9-dodecadienyl acetate in the European grapevine moth, Lobesia botrana, Involving A11 desaturation and an elusive 47 desaturase. Journal of chemical ecology 47: 248-264 (2021).
37. Wang H-L, Lienard M A, Zhao C—H, Wang C-Z and Löfstedt C, Neofunctionalization in an ancestral insect desaturase lineage led to rare 46 pheromone signals in the Chinese tussah silkworm. Insect biochemistry and molecular biology 40: 742-751 (2010).
38. Antony B, Ding B-J, Moto K I, Aldosari S A and Aldawood A S, Two fatty acyl reductases involved in moth pheromone biosynthesis. Scientific reports 6: 1-14 (2016).
39. Ding B-J, Lager I, Bansal S, Durrett T P, Stymne S and Löfstedt C, The yeast ATF1 acetyltransferase efficiently acetylates insect pheromone alcohols: implications for the biological production of moth pheromones. Lipids 51: 469-475 (2016).
40. Atanassov I I, Atanassov I I, Etchells J and Turner S R, A simple, flexible and efficient PCR-fusion/gateway cloning procedure for gene fusion, site-directed mutagenesis, short sequence insertion and domain deletions and swaps. Plant methods 5: 1-11 (2009).
41. Hull J J and Fónagy A. Molecular basis of pheromonogenesis regulation in moths. In Olfactory concepts of insect control-alternative to insecticides. Springer, pp. 151-202 (2019).
42. Sperling P, Ternes P, Zank T and Heinz E, The evolution of desaturases. Prostaglandins, Leukotrienes and Essential Fatty Acids 68: 73-95 (2003).
43. Ding B-J, Hofvander P, Wang H-L, Durrett T P, Stymne S and Löfstedt C, A plant factory for moth pheromone production. Nature communications 5: 1-7 (2014).
44. Moto Ki, Suzuki M G, Hull J J, Kurata R, Takahashi S, Yamamoto M, et al., Involvement of a bifunctional fatty-acyl desaturase in the biosynthesis of the silkmoth, Bombyx mori, sex pheromone. Proceedings of the National Academy of Sciences 101: 8631-8636 (2004).
45. Matoušková P, Pichová I and Svatoš A, Functional characterization of a desaturase from the tobacco hornworm moth (Manduca sexta) with bifunctional Z11- and 10, 12-desaturase activity. Insect biochemistry and molecular biology 37: 601-610 (2007).
46. Liénard M A, Strandh M, Hedenström E, Johansson T and Löfstedt C, Key biosynthetic gene subfamily recruited for pheromone production prior to the extensive radiation of Lepidoptera. BMC Evolutionary Biology 8: 270 (2008).
47. Lienard M A, Lassance J-M, Wang H-L, Zhao C—H, Piškur J, Johansson T, et al., Elucidation of the sex-pheromone biosynthesis producing 5, 7-dodecadienes in Dendrolimus punctatus (Lepidoptera: Lasiocampidae) reveals Δ11- and Δ9-desaturases with unusual catalytic properties. Insect biochemistry and molecular biology 40: 440-452 (2010).
48. Xia Y-H, Zhang Y-N, Ding B-J, Wang H-L and Löfstedt C, Multi-functional desaturases in two Spodoptera moths with Δ11 and Δ12 desaturation activities. Journal of chemical ecology 45: 378-387 (2019).
49. Serra M, Piña B, Bujons J, Camps F and Fabriàs G, Biosynthesis of 10, 12-dienoic fatty acids by a bifunctional Δ11 desaturase in Spodoptera littoralis. Insect biochemistry and molecular biology 36: 634-641 (2006).
50. Lassance J-M, Ding B-J and Löfstedt C, Evolution of the codling moth pheromone via an ancient gene duplication. BMC biology 19: 1-20 (2021).
51. Jurenka R, Biosynthetic pathway for producing the sex pheromone component (Z, E)-9,12-tetradecadienyl acetate in moths involves a Δ12 desaturase. Cellular and Molecular Life Sciences CMLS 53: 501-505 (1997).
52. Henrick C A, The synthesis of insect sex phermones. Tetrahedron 33: 1845-1889 (1977).
53. Xia Y-H, Ding B-J, Wang H-L, Hofvander P, Jarl-Sunesson C and Löfstedt C, Production of moth sex pheromone precursors in Nicotiana spp.: a worthwhile new approach to pest control. Journal of Pest Science 93: 1333-1346 (2020).

The above description is given by way of example, and not limitation. Given the above disclosure, one skilled in the art could devise variations that are within the scope and spirit of the invention disclosed herein, including the use of various species to produce the final ZETA pheromone product. Further, the various features of the embodiments disclosed herein can be used alone, or in varying combinations with each other and are not intended to be limited to the specific combination described herein. Thus, the scope of the claims is not to be limited by the illustrated embodiments.

What is claimed is:

1. A method of producing (Z,E)-9,12-tetradecadienyl acetate, said method comprising:
a) genetically modifying a genome of a species to incorporate into the genome a gene encoding an E12 fatty acyl desaturase; and
b) providing a fatty acid to the species.

2. The method of claim 1, wherein the fatty acid is a fatty acid methyl ester.

3. The method of claim 2, wherein the fatty acid methyl ester is myristic acid methyl ester.

4. The method of claim 1, wherein the gene encoding an E12 fatty acyl desaturase is the gene of SEQ ID NO: 2, and wherein the genome further incorporates a gene encoding a Z9 desaturase, a gene encoding a fatty acyl reductase, and a gene encoding an acetyltransferase.

5. The method of claim 4, wherein the gene encoding a Z9 desaturase is Dmel_D9 from Drosophila melanogaster, the gene encoding a fatty acyl reductase is SexipgFARII from Spodoptera exigua, and the gene encoding an acetyltransferase is ATF1 from Saccharomyces cerevisiae.

6. The method of claim 5, wherein the species is Saccharomyces cerevisiae.

7. A genetically modified yeast having incorporated into the genome a gene encoding an E12 fatty acyl desaturase, wherein the yeast produces a pheromone.

8. The genetically modified yeast of claim 7, wherein the gene encoding an E12 fatty acyl desaturase is the gene of SEQ ID NO: 2.

9. The genetically modified yeast of claim 7, wherein the E12 fatty acyl desaturase is the peptide of SEQ ID NO: 8.

10. The genetically modified yeast of claim 7, wherein the genome further incorporates a gene encoding a Z9 desaturase, a gene encoding a fatty acyl reductase, and a gene encoding an acetyltransferase.

11. The genetically modified yeast of claim 10, wherein the gene encoding a Z9 desaturase is Dmel_D9 from Drosophila melanogaster, the gene encoding a fatty acyl reductase is SexipgFARII from Spodoptera exigua, and the gene encoding an acetyltransferase is ATF1 from Saccharomyces cerevisiae.

12. The genetically modified yeast of claim 7, wherein the yeast is Saccharomyces cerevisiae.

13. The genetically modified yeast of claim 7, wherein the pheromone is (Z,E)-9,12-tetradecadienyl acetate.

14. A genetically modified plant having incorporated into the genome a gene encoding an E12 fatty acyl desaturase, wherein the plant produces a pheromone.

15. The genetically modified plant of claim 14, wherein the gene encoding an E12 fatty acyl desaturase is the gene of SEQ ID NO: 2.

16. The genetically modified plant of claim 14, wherein the E12 fatty acyl desaturase is the peptide of SEQ ID NO: 8.

17. The genetically modified plant of claim 14, wherein the genome further incorporates a gene encoding a Z9 desaturase, a gene encoding a fatty acyl reductase, a gene encoding an acetyltransferase, and a gene encoding a 14-16 carbon fatty acid specific thioesterase.

18. The genetically modified plant of claim 17, wherein the gene encoding a Z9 desaturase is Dmel_D9 from Drosophila melanogaster, the gene encoding a fatty acyl reductase is SexipgFARII from Spodoptera exigua, and the gene encoding an acetyltransferase is ATF1 from Saccharomyces cerevisiae.

19. The genetically modified plant of claim 14, wherein the plant is Nicotiana benthamiana.

20. The genetically modified plant of claim 14, wherein the pheromone is (Z,E)-9,12-tetradecadienyl acetate.


SEQUENCE LISTING (12 sequences)

SEQ ID NO: 1 - RNA, 993 nt, Cadra cautella
  source 1..993: mol_type = mRNA
atggaaaaggacactcaaaacgacgtcactttatgtgactcgcacaaattcaaactcgttaaacacaatgtaataaagtttgcgtatatgcatctagctgcattgtatggagtctacttatgcttcacctcagcgaaatggcaaacactattatggggatttttattgcttgaaggagcgaagattggtatcacggcgggggcgcacagactttggtgtcacagggcgtacaaggccaaaatgccgctgcagattatcctcttgatcttaaatagcatcgcgtttatgaatacagccatttactgggtaagggaccatcgcattcaccacaagtacaccgacacagatgccgaccctcacaactcaagacgaggctttttcttctcccaaataggatggttgttcgttcggaaacaccctcaagttatggaaaaaggaaaaacaatttatatggatgatatttacagtaacaaacttttggtgttccaaagaaagtatgcctttatcgtcatcacgctgtgggcgtatgtaataccggcagttgtcccaatgtatttctggaatgaaagtctcagtaattcgtggcatatttgtacgatgcttcgtcacgttctcaccatcaatatgattttcttggtcaacagcatcgggcataaatggggtaatcgaccttttgataaaagtattaggcctgttgagaatgttgccgtttcaattttgtctacgggagagtgtttccgcaattaccaccacgtctttccctttgattacaaagcgtccgaactcggagacacaatgtttaacgctgctacgattttcatcaatttctttgcctggcttggttgggcttatgacttgaaaataattccggacgagatgattatagcaagatcgaaaagaactggtgatggcaagaacatatggggatggggagacaaagaccaaactgaagaagaaacgaagaatgtaaaactgttatattgtaaaggttag

SEQ ID NO: 2 - RNA, 1017 nt, Cadra cautella
  source 1..1017: mol_type = mRNA
atgactcccaacccagaatctactgccatcttagcagaaggctccgaggatcctcggtttgagaaactagtggccccgcaagctggaccaagaaagtacaagtttgtgttattaaatatgatgtctcatagctactggcacttatcggctatttatggcctttacttgtgcttcacttcagcaaaatttgccaccatgttatttgcactcttcatattcgtgatctcggatataggagtaacagcgggttctcacagactttgggcgcatcgttcttacaaagtcaaactgccattggaaatcctcttaatcataatgaactctatagcataccaagacacggccttcacttgggcgagagatcaccgacttcaccatcgatacgctgatacggatgctgatccacacaattctaccagaggattcttctactctcatattggttggcttttcgtgagaaaacaccatgaagtcatagctagagggaaaacaatcccgatagatgatctgaagaaaaacaaggtgctccaattccaaagaaaatatgcgtatataatcatgagcacgttatgtttttttatgccgaccttcgtgccagtctacttctggggcgagagtatcagcaccgcctggcatgtcaatttgctgcgatacgtctgtaacctcaacgcgatattcttagtaaacagcgcttcccatatgttcggctacaagccatatgataagaacatcgcctcagtacagaatatccctgtcaccttcgtaacattaggagagggttaccacaactaccaccatacatacccctgggactacaaagcggcggaattgggaaacaacaagttcaatttgacaacaaaattcattgattttttcgcttggattggctgggcttacgatttgaaaactgttccccaagaatctgtatacaagagaatggagaggactggtgatggaacaaacttgtggggctatgaaagagaaaatattgaaaatgaagtaaacagtgatttttga

SEQ ID NO: 3 - RNA, 1281 nt, Cadra cautella
  source 1..1281: mol_type = mRNA
acatgggggtagtgtgtttccgaattttgtttgaaacacttgtaatagacgacgctaataaaacttagtgtttagtttgtcgtgaactaccagcgaagtgccaattggatttaagtacttcacaactattctaaaaaaatgccacctcaaggtcaagagcgagagtcctgggtgctgtacgagacagacgagaaggccgcgggtgatgccaccctggtagttccgccctccgcagacaagagagagtggaaaatagtctggagaaatgtcatcctgtttgctcttctacacatcggtggcgtgtatggtgcttaccttttcctcttcaaagccatgtggacgactgggttgttcgcattcttcctgtacctgtgctctgggctgggcatcacggccggcgcccaccgtctgtgggctcacaagtcgtacaaggctcgtttgccgctgcgggtcctactgacggtcttcaacacgctagctttccaggatgccgtgattgactgggcgcgcgaccaccgcctgcaccacaagtactcagagaccgacgccgacccccacaacgccacccgcggcttcttcttctcacacgtgggctggctgctcgtgaggaagcacccgcagatcaaggagaagggctccaccatcgatctcagcgacttgaaggccgatcccgttctgcatttccagaaaaagtactacctgattctcatgccgttgacctgtttcattctgcccacgtatatcccaactctgtggggcgagtcactctggaacgcgtacttcgtatgcgcgatattcagatacgtgtacgttctcaacgtgacctggctggtcaactccgcagcacatgcctggggcagcaagccttacgacaagaacatcaaccccgtagaaaccaggcccgtgtcccttgtggtcctcggagaagggttccacaactaccaccacaccttcccttgggattacaagactgctgagctaggagattactctttgaacttctctaagttgtttatcgatgccatggcgaagatcggttgggcttacgacctgaagaccgtctcttcggacgtggttgaggcgcgcgtgaagcggacgggcgacggcagccaccacgtgtggggctgggacgacaaggacgtcccagcgcaagagaaggaaggcgctactatcatcaacccgtccaaaagtgaataattcacagcacaagcttatagggaactaatttttagagcttcctacattttagatacaaaaaaaaaaaaaaaaaaaaaaaaaaaa

SEQ ID NO: 4 - RNA, 1447 nt, Cadra cautella
  source 1..1447: mol_type = mRNA
aggttttatcttgatacattgattaaaaactgagaccatgagtgtgagaaattaacagcagcaaatcagcaaattacacgcagacgaagtccgagagcacaggctgttagaaaataaaatacaaaatactgtcaatctataatttgattaaaacctgtgaacatggctccaaacgctacagatgccaacggggttctctttgaaactgacgcagctactccagacctggccctaccccacaccccagttcagcaagcagacgactacccgaggaaatatgtctggagaaatataatattattcgcatacctgcatattgctgctatatacggtggctacctattcctattccatgctaaatggcaaaccgatatatttgcttacatactttatgtgttctccggattggggatcaccgcaggggcccaccgcctctgggcccacaaatcctacaaagctaaatggccactcagactgatccttgtcgccttcaatactctggcgtttcaggactctgccatagactgggcgcgcgaccacagaatgcaccacaagtactcagagacggacgccgaccctcacaacgcgactcgcggcttcttcttctcccacattggctggctgctggttaggaagcaccctgaacttaagaggaaaggcaaagggctggaccttagcgatttatatgctgaccctatcctccgattccaaaagaaatactacctgatcttgatgccgctgacttgcttcgtgctgcccaccgtggtgcccgtgtactactggggcgagacctggaccaacgccttcctcgtggccgctctattccggtatgccttcatcctcaatgtcacttggctcgtcaactccgcggcccacaagtggggcgacaaaccctacgacaaggccatcaagccttccgaaaacatttccgtttccgtattcgctcttggagaaggctttcacaactaccaccacaccttcccctgggactacaagacggccgaactcggcaacaacagactaaacttcactaccaatttcatcaatttctttgccaaaatcggatgggcgtatgaccttaagacggtttctaatgaaatcattaagaccagggctcaacgaactggcgatggctcgcatcatctttggggttggggcgataaagaccattcgcgcgaagaaatggatgccgctattagattgcacccaaaagacgactaaaacttgtctattgttaatgagattatagatctatgtagcacattacgaaactttcacatctcacacgtttgattcagcaaatcaatctgaatacattttttgaaatttgtttaacaaggcatcttcgttattgaaagattaattaacatttagttaaggactttccaaaatgatttgaaagtttttttaaaaaacaatagaatgacgaatttattaacatgcgctt

SEQ ID NO: 5 - RNA, 1041 nt, Cadra cautella
  source 1..1041: mol_type = mRNA
ttcgtcatggttccgtataagggttctagtaccgttttgacagaggatcagcctcagtttgaaaaactggtcgcccctcaagctggtccgaggaagtatgagatagtatacaggaaccttattacatttggatactggcacctagcagctatttatgggctctacttgtgctttacgaccgctaaatgggctacaattatatttgccttgttcttatatactatagcggagataggaataaccgctggtgcgcatagactatggacacaccgggcttacaaagctaagcttcctttacaaatccttttaatggtaatgaatagtattgccttccaagacactgctctaacgtggtgtcgcgatcatcgcatgcaccacagatattctgacactgatgcggatccccataatgcgacgagagggttcttctactcccacgtaggttggcttctggtgaagaagcaccctgaagtcaaagcaagaggaaaatacatcccattagacgatttattaaataatccagtgctaagatttcagaaaaaatacgccgtccctgtcgtgggcacgttgtgtttcctcatgcctacgtttgtgccggtctacttctggggagaaaccatcagcactgcatggcatattaacttcctgcgatacgtcatgaacctcaacatgactttccttgtcaatagcgccgcccacatgtttggaaataaaccctacgatagaactctagtgccagtacaaaacgttgcagtaagctttgctacatttggcgagggtttccataattatcaccacacttatccttgggactaccgaacggcggagttaggaaacaataaactgaatataacaactcattttatagactttttcgcgtggatcggctgggcttatgacttgaaaacaattcctcaagatgctattgaaaagagaatgatcaggaccggtgacggaactgatatgtggggctttggagagaaaagatttcgaaaagaggttgataattctaatgaaacctatagtgacttttgatctact

SEQ ID NO: 6 - RNA, 1035 nt, Cadra cautella
  source 1..1035: mol_type = mRNA
atggttccgtataaggattccgatgaggttctgacagaagatcagcctcagttcgaaaaattggtggctcctcaagctggtccgaggaagtacaagatagtatacaggaaccttataacatttggatactggcacctagcagctatttatgggctctacttgtgcttcacgaccgctaaatgggctacagttatatttgccatcttcttatataccatggcggagatcggaataaccgctggtgcgcatagactgtggactcaccgggcttacaaagctaagcttcctttacaaatccttttaatggtaatgaacagtattgccttccaagacactgccctaacgtggtgtcgcgatcatcgcttgcaccacagatactccgacactgacgctgatccccacaatgcggcgagagggttcttctactcccacgtgggttggctcctggtgaggaaacaccctgaagtcaaagccagaggaaaatacaccccattagatgatttactaaataatccggtgctgaaatttcagaagaaatacgccattccattcgtgggcacgatgtgttttctgatgcctacgcttgtccctgtctacttttggggcgaaaacatctacactgcatggaatatcaacctcttgcgatatgtcatgaatctcaatatgactttcctagtcaatagcgccgctcacatgtttggaaataaaccctatgatacatctcttctgccagttcaaaaccttgcagtaagcttcgctacatttggcgagggtttccataattatcaccatacttacccttgggactaccgaacagcggagttaggaaacaacaaactgaatatcacaactctgtttatagattttttcgcgtggatcggctgggcttatgacttgaaaacaattcctcgagatgctattgaaaagaaaaaggccaggaccggtgacggaactgatatgtggggctttggaaagaaaagatcccaaaaagaagtaaataattctactgaaaccaataatgacttggataattaa

SEQ ID NO: 7 - protein, 330 aa, Cadra cautella
MEKDTQNDVTLCDSHKFKLVKHNVIKFAYMHLAALYGVYLCFTSAKWQTLLWGFLLLEGAKIGITAGAHRLWCHRAYKAKMPLQIILLILNSIAFMNTAIYWVRDHRIHHKYTDTDADPHNSRRGFFFSQIGWLFVRKHPQVMEKGKTIYMDDIYSNKLLVFQRKYAFIVITLWAYVIPAVVPMYFWNESLSNSWHICTMLRHVLTINMIFLVNSIGHKWGNRPFDKSIRPVENVAVSILSTGECFRNYHHVFPFDYKASELGDTMFNAATIFINFFAWLGWAYDLKIIPDEMIIARSKRTGDGKNIWGWGDKDQTEEETKNVKLLYCKG

SEQ ID NO: 8 - protein, 338 aa, Cadra cautella
MTPNPESTAILAEGSEDPRFEKLVAPQAGPRKYKFVLLNMMSHSYWHLSAIYGLYLCFTSAKFATMLFALFIFVISDIGVTAGSHRLWAHRSYKVKLPLEILLIIMNSIAYQDTAFTWARDHRLHHRYADTDADPHNSTRGFFYSHIGWLFVRKHHEVIARGKTIPIDDLKKNKVLQFQRKYAYIIMSTLCFFMPTFVPVYFWGESISTAWHVNLLRYVCNLNAIFLVNSASHMFGYKPYDKNIASVQNIPVTFVTLGEGYHNYHHTYPWDYKAAELGNNKFNLTTKFIDFFAWIGWAYDLKTVPQESVYKRMERTGDGTNLWGYERENIENEVNSDF

SEQ ID NO: 9 - protein, 352 aa, Cadra cautella
MPPQGQERESWVLYETDEKAAGDATLVVPPSADKREWKIVWRNVILFALLHIGGVYGAYLFLFKAMWTTGLFAFFLYLCSGLGITAGAHRLWAHKSYKARLPLRVLLTVFNTLAFQDAVIDWARDHRLHHKYSETDADPHNATRGFFFSHVGWLLVRKHPQIKEKGSTIDLSDLKADPVLHFQKKYYLILMPLTCFILPTYIPTLWGESLWNAYFVCAIFRYVYVLNVTWLVNSAAHAWGSKPYDKNINPVETRPVSLVVLGEGFHNYHHTFPWDYKTAELGDYSLNFSKLFIDAMAKIGWAYDLKTVSSDVVEARVKRTGDGSHHVWGWDDKDVPAQEKEGATIINPSKSE

SEQ ID NO: 10 - protein, 352 aa, Cadra cautella
MAPNATDANGVLFETDAATPDLALPHTPVQQADDYPRKYVWRNIILFAYLHIAAIYGGYLFLFHAKWQTDIFAYILYVFSGLGITAGAHRLWAHKSYKAKWPLRLILVAFNTLAFQDSAIDWARDHRMHHKYSETDADPHNATRGFFFSHIGWLLVRKHPELKRKGKGLDLSDLYADPILRFQKKYYLILMPLTCFVLPTVVPVYYWGETWTNAFLVAALFRYAFILNVTWLVNSAAHKWGDKPYDKAIKPSENISVSVFALGEGFHNYHHTFPWDYKTAELGNNRLNFTTNFINFFAKIGWAYDLKTVSNEIIKTRAQRTGDGSHHLWGWGDKDHSREEMDAAIRLHPKDD

SEQ ID NO: 11 - protein, 342 aa, Cadra cautella
MVPYKGSSTVLTEDQPQFEKLVAPQAGPRKYEIVYRNLITFGYWHLAAIYGLYLCFTTAKWATIIFALFLYTIAEIGITAGAHRLWTHRAYKAKLPLQILLMVMNSIAFQDTALTWCRDHRMHHRYSDTDADPHNATRGFFYSHVGWLLVKKHPEVKARGKYIPLDDLLNNPVLRFQKKYAVPVVGTLCFLMPTFVPVYFWGETISTAWHINFLRYVMNLNMTFLVNSAAHMFGNKPYDRTLVPVQNVAVSFATFGEGFHNYHHTYPWDYRTAELGNNKLNITTHFIDFFAWIGWAYDLKTIPQDAIEKRMIRTGDGTDMWGFGEKRFRKEVDNSNETYSDF

SEQ ID NO: 12 - protein, 344 aa, Cadra cautella
MVPYKDSDEVLTEDQPQFEKLVAPQAGPRKYKIVYRNLITFGYWHLAAIYGLYLCFTTAKWATVIFAIFLYTMAEIGITAGAHRLWTHRAYKAKLPLQILLMVMNSIAFQDTALTWCRDHRLHHRYSDTDADPHNAARGFFYSHVGWLLVRKHPEVKARGKYTPLDDLLNNPVLKFQKKYAIPFVGTMCFLMPTLVPVYFWGENIYTAWNINLLRYVMNLNMTFLVNSAAHMFGNKPYDTSLLPVQNLAVSFATFGEGFHNYHHTYPWDYRTAELGNNKLNITTLFIDFFAWIGWAYDLKTIPRDAIEKKKARTGDGTDMWGFGKKRSQKEVNNSTETNNDLDN